(19) United States
(12) Patent Application Publication
(10) Pub. No.: US 2005/0042731 A1
(43) Pub. Date: Feb. 24, 2005

(54) Secreted proteins

(76) Inventors: Richard Joseph Fagan, London (GB); Christopher Benjamin Phelps, London (GB); Alex Gutteridge, Haslingfield (GB); Christine Power, Thoiry (FR)

Correspondence Address: FROMMER LAWRENCE & HAUG, 745 FIFTH AVENUE- 10TH FL., NEW YORK, NY, 10151, US

(21) Appl. No.: 10/873,332

(22) Filed: Jun. 21, 2004

Related U.S. Application Data
(63) Continuation-in-part of application No. PCT/GB02/05890, filed on Dec. 23, 2002.

(30) Foreign Application Priority Data
Dec. 21, 2001 (GB) 0130720.6

(51) Int. Cl. C07K 14/52; C07H 21/04; C12P 21/02

(52) U.S. Cl. 435/69.5; 435/320.1; 435/325; 530/351; 536/23.5

(57) ABSTRACT

This invention relates to novel proteins (INSP032, INSP033, INSP034, INSP036, INSP038), herein identified as members of the four helical bundle cytokine family and to the use of these proteins and nucleic acid sequences from the encoding genes in the diagnosis, prevention and treatment of disease.

### REFERENCE TO RELATED APPLICATIONS

This application is a continuation-in-part of International Patent Application PCT/GB02/05890 filed Dec. 23, 2003 and published as WO 03/055912 on Jul. 10, 2003, which claims priority from Great Britain Application Number 0130720.6 filed Dec. 21, 2001. Each of these applications, and each application and patent mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

It is noted that in this disclosure, terms such as “comprises”, “comprised”, “comprising”, “contains”, “containing” and the like can have the meaning attributed to them in U.S. Patent law; e.g., they can mean “includes”, “included”, “including” and the like. Terms such as “consisting essentially of” and “consists essentially of” have the meaning attributed to them in U.S. Patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. Patent law; namely, that these terms are closed ended.

### SUMMARY OF THE INVENTION

This invention relates to novel proteins (INSP032, INSP033, INSP034, INSP036, INSP038), herein identified as secreted proteins (in particular, as members of the four helical bundle cytokine family) and to the use of these proteins and nucleic acid sequences from the encoding genes in the diagnosis, prevention and treatment of disease.

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

### SECRETED PROTEIN BACKGROUND

The ability of cells to make and secrete extracellular proteins is central to many biological processes. Enzymes, growth factors, extracellular matrix proteins and signalling molecules are all secreted by cells. This is through fusion of a secretory vesicle with the plasma membrane. In most cases, but not all, proteins are directed to the endoplasmic reticulum and into secretory vesicles by a signal peptide. Signal peptides are cis-acting sequences that affect the transport of polypeptide chains from the cytoplasm to a membrane bound compartment such as a secretory vesicle. Polypeptides that are targeted to the secretory vesicles are either secreted into the extracellular matrix or are retained in the plasma membrane. The polypeptides that are retained in the plasma membrane will have one or more transmembrane domains. Examples of secreted proteins that play a central role in the functioning of a cell are cytokines, hormones, extracellular matrix proteins (adhesion molecules), proteases, and growth and differentiation factors. Description of some of the properties of these proteins follows.

### INTRODUCTION TO CYTOKINES

Cytokines are a family of growth factors secreted primarily from leukocytes, and are messenger proteins that act as potent regulators capable of effecting cellular processes at sub-nanomolar concentrations. Interleukins, neurotrophins, growth factors, interferons and chemokines all define cytokine families that work in conjunction with cellular receptors to regulate cell proliferation and differentiation. Their size allows cytokines to be quickly transported around the body and degraded when required. Their role in controlling a wide range of cellular functions, especially the immune response and cell growth has been revealed by extensive research over the last twenty years (Boppana, S.B (1996) Indian. J. Pediatr. 63(4):447-52). Cytokines, as for other growth factors, are differentiated from classical hormones by the fact that they are produced by a number of different cell types rather than just one specific tissue or gland, and also effect a broad range of cells via interaction with specific high affinity receptors located on target cells.

All cytokine communication systems show both pleiotropy (one messenger producing multiple effects) and redundancy (each effect is produced by more than one messenger (Tringali, G. et al (2000) Therapie. 55(1):171-5; Tessarollo, L. (1998) Cytokine Growth Factor Rev. 9(2):125-137). An individual cytokine's effects on a cell can also be dependent on its concentration, the concentration of other cytokines, the temporal sequence of cytokines, and the internal state of the cell (cell cycle, presence of neighbouring cells, cancerous).

Although cytokines are typically small (under 200 amino acids) proteins they are often formed from larger precursors which are post-translationally spliced. This, in addition to mRNA alternative splicing pathways, give a wide spectrum of variants of each cytokine each of which may differ substantially in biological effect. Membrane and extracellular matrix associated forms of many cytokines have also been isolated (Okada-Ban, M. et al (2000) Int. J. Biochem. Cell Biol. 32(3):263-267; Atamas, S.P. (1997) Life Sci. 61(12):1105-1112).

Cytokines can be grouped into families, though most are unrelated. Categorisation is usually based on secondary structure composition, as sequence similarity is often very low. The families are named after the archetypal member e.g. IFN-like, IL2-like, IL1-like, I1-6 like and TNF-like (Zlotnik, A. et al., (2000) Immunity. 12(2):121-127).

Studies have shown cytokines are involved in many important reactions in multi-cellular organisms such as immune response regulation (Nishihira, J. (1998) Int. J. Mol. Med. 2(1):17-28), inflammation (Kim, P.K. et al, (2000) Surg. Clin. North. Am. 80(3):885-894), wound healing (Clark, R.A. (1991) J. Cell Biochem. 46(1):1-2), embryogenesis and development, and apoptosis (Flad, H.D. et al, (1999) Pathobiology. 67(5-6):291-293). Pathogenic organisms (both viral and bacterial) such as HIV and Kaposi's sarcoma-associated virus encode anti-cytokine factors as well as cytokine analogues, which allow them to interact with cytokine receptors and control the body's immune response (Sozzani, S. et al., (2000) Pharm. Acta. Helv. 74(2-3):305-312; Aoki, Y. et al., (2000) J. Hematother. Stem Cell Res. 9(2):137-145). Virally encoded cytokines, virokines, have been shown to be required for pathogenicity of viruses due to their ability to mimic and subvert the host immune system.

Cytokines may be useful for the treatment, prevention and/or diagnosis of medical conditions and diseases which include immune disorders, such as autoimmune disease, rheumatoid arthritis, osteoarthritis, psoriasis, systemic lupus erythematosus, and multiple sclerosis, inflammatory disorders, such as allergy, rhinitis, conjunctivitis, glomerulonephritis, uveitis, Crohn's disease, ulcerative colitis, inflammatory bowel disease, pancreatitis, digestive system inflammation, sepsis, endotoxic shock, septic shock, cachexia, myalgia, ankylosing spondylitis, myasthenia gravis, post-viral fatigue syndrome, pulmonary disease, respiratory distress syndrome, asthma, chronic-obstructive pulmonary disease, airway inflammation, wound healing, endometriosis, dermatological disease, Behcet's disease, neoplastic disorders, such as melanoma, sarcoma, renal tumour, colon tumour, haematological disease, myeloproliferative disorder, Hodgkin's disease, osteoporosis, obesity, diabetes, gout, cardiovascular disorders, reperfusion injury, atherosclerosis, ischaemic heart disease, cardiac failure, stroke, liver disease, AIDS, AIDS related complex, neurological disorders, male infertility, ageing and infections, including plasmodium infection, bacterial infection and viral infection, particularly human herpesvirus 5 (cytomegalovirus) infection.

It has been shown that the viral-encoded cytokine, macrophage inhibitory protein-II is able to mediate selective recruitment of Th2-type cells and evasion from a cytotoxic immune response (Weber KS et al., (2001), Eur J Immunol. 2001 31(8):2458-66). These data provide evidence for an immunomodulatory role of vMIP-II in directing inflammatory cell recruitment away from a Th1-type towards a Th2-type response and thereby facilitating evasion from cytotoxic reactions. This protein could therefore be used to modulate diseases in which over-stimulation of the Th1-type immune response is implicated, such as irritable bowel syndrome. In another study, Kawamoto S et al., (Int Immunol. 2001 13(5):685-94) presented results that indicate that vIL-10 may well be superior to cellular IL-10 in the treatment of autoimmune diabetes. These results indicate that viral-encoded cytokines have potential therapeutic benefit beyond viral clearance alone.

Clinical use of cytokines has focused on their role as regulators of the immune system (Rodriguez, F.H. et al., (2000) Curr. Pharm. Des. 6(6):665-680) for instance in promoting a response against thyroid cancer (Schmutzler, C. et al., (2000) 143(1):15-24). Their control of cell growth and differentiation has also made cytokines anti-cancer targets (Lazar-Molnar, E. et al., (2000) Cytokine. 12(6):547-554; Gado, K. (2000) 24(4):195-209). Novel mutations in cytokines and cytokine receptors have been shown to confer disease resistance in some cases (van Deventer, S.J. et al., (2000) Intensive Care Med. 26 (Suppl 1):S98:S102). The creation of synthetic cytokines (muteins) in order to modulate activity and remove potential side effects has also been an important avenue of research (Shanafelt, A.B. et al., (1998) 95(16):9454-9458).

As described above, cytokine molecules have been shown to play a role in diverse physiological functions, many of which can play a role in disease processes. Alteration of their activity is a means to alter the disease phenotype and as such identification of novel cytokine molecules is highly relevant as they may play a role in or be useful in the development of treatments for the diseases identified above, as well as other disease states.

### THE INVENTION

The invention is based on the discovery that the INSP032, INSP033, INSP034, INSP036, INSP038 proteins function as secreted protein molecules and, moreover, as secreted proteins in the four helical bundle cytokine class.

In one embodiment of the first aspect of the invention, there is provided a polypeptide, which polypeptide:
(i) comprises the amino acid sequence as recited in SEQ ID NO:2 and/or SEQ ID NO:4;
(ii) is a fragment thereof having secreted protein function, and in particular having four helical bundle cytokine function or having an antigenic determinant in common with the polypeptides of (i); or
(iii) is a functional equivalent of (i) or (ii).

Preferably, the polypeptide according to this embodiment comprises the amino acid sequence recited in SEQ ID NO:6. More preferably, the polypeptide consists of the amino acid sequence recited in SEQ ID NO:6.

The polypeptide having the sequence recited in SEQ ID NO:2 is referred to hereafter as “the INSP032 exon 1 polypeptide”. The polypeptide having the sequence recited in SEQ ID NO:4 is referred to hereafter as “the INSP032 exon 2 polypeptide”. Combining SEQ ID NO:2 and SEQ ID NO:4 produces the sequence recited in SEQ ID NO:6. SEQ ID NO:6 is referred to hereafter as “the INSP032 polypeptide”.

In a second embodiment of the first aspect of the invention, there is provided a polypeptide, which polypeptide:
(i) comprises the amino acid sequence as recited in SEQ ID NO:8;
(ii) is a fragment thereof having secreted protein function, and in particular, having four helical bundle cytokine function, more particularly, having granulocyte colony-stimulating factor activity, or having an antigenic determinant in common with the polypeptides of (i); or
(iii) is a functional equivalent of (i) or (ii).

The polypeptide having the sequence recited in SEQ ID NO:8 is referred to hereafter as “the INSP033 exon 1 polypeptide” or the “INSP033 polypeptide”. INSP033 is also referred to as IPAAA24020. A polypeptide defined by the “working draft sequence” disclosed as sequence accession number AC021857 on the NCBI database is specifically excluded from the scope of the present invention.

Preferably, a polypeptide according to the second embodiment of this aspect of the invention possesses secreted protein function, four helical bundle cytokine function in particular, and granulocyte colony-stimulating factor activity more particularly.

In a third embodiment of the first aspect of the invention, there is provided a polypeptide, which polypeptide:
(i) comprises the amino acid sequence as recited in SEQ ID NO:10, SEQ ID NO:12 and/or SEQ ID NO:14;
(ii) is a fragment thereof having secreted protein function, and in particular having four helical bundle cytokine function or having an antigenic determinant in common with the polypeptides of (i); or
(iii) is a functional equivalent of (i) or (ii).

Preferably, the polypeptide according to this embodiment comprises the amino acid sequence recited in SEQ ID NO:16. More preferably, the polypeptide consists of the amino acid sequence recited in SEQ ID NO:16.

The polypeptide having the sequence recited in SEQ ID NO:10 is referred to hereafter as “the INSP034 exon 1 polypeptide”. The polypeptide having the sequence recited in SEQ ID NO:12 is referred to hereafter as “the INSP034 exon 2 polypeptide”. The polypeptide having the sequence recited in SEQ ID NO:14 is referred to hereafter as “the INSP034 exon 3 polypeptide”. Combining SEQ ID NO:10, SEQ ID NO:12 and SEQ ID NO:14 produces the sequence recited in SEQ ID NO:16. SEQ ID NO:16 is referred to hereafter as “the INSP034 polypeptide”.

In a fourth embodiment of the first aspect of the invention, there is provided a polypeptide, which polypeptide:
(i) comprises the amino acid sequence as recited in SEQ ID NO:24, SEQ ID NO:26, SEQ ID NO:28, SEQ ID NO:30, SEQ ID NO:32 and/or SEQ ID NO:34;
(ii) is a fragment thereof having secreted protein function, and in particular having four helical bundle cytokine function or having an antigenic determinant in common with the polypeptides of (i); or
(iii) is a functional equivalent of (i) or (ii).

Preferably, the polypeptide according to this embodiment comprises the amino acid sequence recited in SEQ ID NO:34. More preferably, the polypeptide consists of the amino acid sequence recited in SEQ ID NO:34.

The polypeptide having the sequence recited in SEQ ID NO:24 is referred to hereafter as “the INSP036 exon 1 polypeptide”. The polypeptide having the sequence recited in SEQ ID NO:26 is referred to hereafter as “the INSP036 exon 2 polypeptide”. The polypeptide having the sequence recited in SEQ ID NO:28 is referred to hereafter as “the INSP036 exon 3 polypeptide”. The polypeptide having the sequence recited in SEQ ID NO:30 is referred to hereafter as “the INSP036 exon 4 polypeptide”. The polypeptide having the sequence recited in SEQ ID NO:32 is referred to hereafter as “the INSP036 exon 5 polypeptide”. Combining SEQ ID NO:24, SEQ ID NO:26 and SEQ ID NO:28, SEQ ID NO:30 and SEQ ID NO:32 produces the sequence recited in SEQ ID NO:34. SEQ ID NO:34 is referred to hereafter as “the INSP036 polypeptide”.

In a fifth embodiment of the first aspect of the invention, there is provided a polypeptide, which polypeptide:
(i) comprises the amino acid sequence as recited in SEQ ID NO:38, SEQ ID NO:40, SEQ ID NO:42, SEQ ID NO:44, SEQ ID NO:46 and/or SEQ ID NO:48;
(ii) is a fragment thereof having secreted protein function, and in particular having four helical bundle cytokine function or having an antigenic determinant in common with the polypeptides of (i); or
(iii) is a functional equivalent of (i) or (ii).

Preferably, the polypeptide according to this embodiment comprises the amino acid sequence recited in SEQ ID NO:48. More preferably, the polypeptide consists of the amino acid sequence recited in SEQ ID NO:48.

The polypeptide having the sequence recited in SEQ ID NO:38 is referred to hereafter as “the INSP038 exon 1 polypeptide”. The polypeptide having the sequence recited in SEQ ID NO:40 is referred to hereafter as “the INSP038 exon 2 polypeptide”. The polypeptide having the sequence recited in SEQ ID NO:42 is referred to hereafter as “the INSP038 exon 3 polypeptide”. The polypeptide having the sequence recited in SEQ ID NO:44 is referred to hereafter as “the INSP038 exon 4 polypeptide”. The polypeptide having the sequence recited in SEQ ID NO:46 is referred to hereafter as “the INSP038 exon 5 polypeptide”. Combining SEQ ID NO:38, SEQ ID NO:40 and SEQ ID NO:42, SEQ ID NO:44 and SEQ ID NO:46 produces the sequence recited in SEQ ID NO:48. SEQ ID NO:48 is referred to hereafter as “the INSP038 polypeptide”.

In a second aspect, the invention provides a purified nucleic acid molecule which encodes a polypeptide of the first aspect of the invention. Preferably, the purified nucleic acid molecule has the nucleic acid sequence as recited in SEQ ID NO:1 (encoding the INSP032 exon 1 polypeptide), SEQ ID NO:3 (encoding the INSP032 exon 2 polypeptide), SEQ ID NO:7 (encoding the INSP033 exon 1 polypeptide), SEQ ID NO:9 (encoding the INSP034 exon 1 polypeptide), SEQ ID NO:11 (encoding the INSP034 exon 2 polypeptide), SEQ ID NO:13 (encoding the INSP034 exon 3 polypeptide), SEQ ID NO:23 (encoding the INSP036 exon 1 polypeptide), SEQ ID NO:25 (encoding the INSP036 exon 2 polypeptide), SEQ ID NO:27 (encoding the INSP036 exon 3 polypeptide), SEQ ID NO:29 (encoding the INSP036 exon 4 polypeptide), SEQ ID NO:31 (encoding the INSP036 exon 5 polypeptide), SEQ ID NO:37 (encoding the INSP038 exon 1 polypeptide), SEQ ID NO:39 (encoding the INSP038 exon 2 polypeptide), SEQ ID NO:41 (encoding the INSP038 exon 3 polypeptide), SEQ ID NO:43 (encoding the INSP038 exon 4 polypeptide), SEQ ID NO:45 (encoding the INSP038 exon 5 polypeptide), or is a redundant equivalent or fragment of either of these sequences. Combining SEQ ID NO:1 and SEQ ID NO:3 produces the sequence recited in SEQ ID NO:5. Combining SEQ ID NO:9, SEQ ID NO:11 and SEQ ID NO:13 produces the sequence recited in SEQ ID NO:15. Combining SEQ ID NO:23, SEQ ID NO:25, SEQ ID NO:27, SEQ ID NO:29 and SEQ ID NO:31 produces the sequence recited in SEQ ID NO:33. Combining SEQ ID NO:37, SEQ ID NO:39, SEQ ID NO:41, SEQ ID NO:43 and SEQ ID NO:45 produces the sequence recited in SEQ ID NO:47. Preferably, the purified nucleic acid molecule has the nucleic acid sequence as recited in SEQ ID NO:5 (encoding the INSP032 polypeptide) or the nucleic acid sequence as recited in SEQ ID NO:7 (encoding the INSP033 polypeptide) or the nucleic acid sequence as recited in SEQ ID NO:15 (encoding the INSP034 polypeptide) or the nucleic acid sequence as recited in SEQ ID NO:33 (encoding the INSP036 polypeptide) or the nucleic acid sequence as recited in SEQ ID NO:47 (encoding the INSP038 polypeptide) or is a redundant equivalent or fragment of either of these sequences.

In a third aspect, the invention provides a purified nucleic acid molecule which hybridizes under high stringency conditions with a nucleic acid molecule of the second aspect of the invention.

In a fourth aspect, the invention provides a vector, such as an expression vector, that contains a nucleic acid molecule of the second or third aspect of the invention. Preferred vectors include the PCRII-TOPO-IPAAA24020 and pEAK12D-IPAAA24020-6HIS vectors (see FIGS. 13 and 14).

In a fifth aspect, the invention provides a host cell transformed with a vector of the fourth aspect of the invention.

In a sixth aspect, the invention provides a ligand which binds specifically to, and which preferably inhibits the activity of, the secreted protein activity of, and in particular the four helical bundle cytokine activity, of a polypeptide of the first aspect of the invention. Even more particularly, the invention provides a ligand which binds specifically to, and which preferably inhibits the granulocyte colony-stimulating factor activity of an INSP033 polypeptide of the first aspect of the invention.

In a seventh aspect, the invention provides a compound that is effective to alter the expression of a natural gene which encodes a polypeptide of the first aspect of the invention or to regulate the activity of a polypeptide of the first aspect of the invention.

A compound of the seventh aspect of the invention may either increase (agonise) or decrease (antagonise) the level of expression of the gene or the activity of the polypeptide. Importantly, the identification of the function of the INSP032, INSP033, INSP034, INSP036 and INSP038 exon polypeptides and the INSP032, INSP033, INSP034, INSP036 and INSP038 polypeptides allows for the design of screening methods capable of identifying compounds that are effective in the treatment and/or diagnosis of disease.

In an eighth aspect, the invention provides a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, for use in therapy or diagnosis. These molecules may also be used in the manufacture of a medicament for the treatment of cell proliferative disorders, autoimmune/inflammatory disorders, cardiovascular disorders, neurological disorders, developmental disorders, metabolic disorders, infections and other pathological conditions. Preferably, the disorders include, but are not limited to immune disorders, such as autoimmune disease, rheumatoid arthritis, osteoarthritis, psoriasis, systemic lupus erythematosus, and multiple sclerosis, inflammatory disorders, such as allergy, rhinitis, conjunctivitis, glomerulonephritis, uveitis, Crohn's disease, ulcerative colitis, inflammatory bowel disease, pancreatitis, digestive system inflammation, sepsis, endotoxic shock, septic shock, cachexia, myalgia, ankylosing spondylitis, myasthenia gravis, post-viral fatigue syndrome, pulmonary disease, respiratory distress syndrome, asthma, chronic-obstructive pulmonary disease, airway inflammation, wound healing, endometriosis, dermatological disease, Behcet's disease, neoplastic disorders, such as melanoma, sarcoma, renal tumour, colon tumour, haematological disease, myeloproliferative disorder, Hodgkin's disease, osteoporosis, obesity, diabetes, gout, cardiovascular disorders, reperfusion injury, atherosclerosis, ischaemic heart disease, cardiac failure, stroke, liver disease, AIDS, AIDS related complex, neurological disorders, male infertility, ageing and infections, including plasmodium infection, bacterial infection and viral infection, particularly human herpesvirus 5 (cytomegalovirus) infection, even more particularly, hematological disease, leukocyte disorders including leukopenia, drug-induced leukopenia and leukemia, bone marrow transplantation, bone marrow disease, wound healing, immune disorders such as graft versus host disease and Crohn's disease, neoplastic disorders, melanoma, solid tumour, hyperlipidemia, hyperphosphatemia, anemia, ischemia, stroke, vascular disease, thrombosis, thromboembolism, infarction and infection, most particularly fungal and bacterial infection.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of a polypeptide of the first aspect of the invention as secreted protein molecules. For the INSP033 polypeptide, this aspect of the invention provides for the use of the polypeptide as a secreted protein molecule, preferably a four helical bundle cytokine, and more particularly, a polypeptide with granulocyte colony-stimulating factor activity.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention, for use in the manufacture of a medicanent for the diagnosis or treatment of a disease, such as cell proliferative disorders, autoimmune/inflammatory disorders, cardiovascular disorders, neurological disorders, developmental disorders, metabolic disorders, infections and other pathological conditions. Preferably, these diseases include immune disorders, such as autoimmune disease, rheumatoid arthritis, osteoarthritis, psoriasis, systemic lupus erythematosus, and multiple sclerosis, inflammatory disorders, such as allergy, rhinitis, conjunctivitis, glomerulonephritis, uveitis, Crohn's disease, ulcerative colitis, inflammatory bowel disease, pancreatitis, digestive system inflammation, sepsis, endotoxic shock, septic shock, cachexia, myalgia, ankylosing spondylitis, myasthenia gravis, post-viral fatigue syndrome, pulmonary disease, respiratory distress syndrome, asthma, chronic-obstructive pulmonary disease, airway inflammation, wound healing, endometriosis, dermatological disease, Behcet's disease, neoplastic disorders, such as melanoma, sarcoma, renal tumour, colon tumour, haematological disease, myeloproliferative disorder, Hodgkin's disease, osteoporosis, obesity, diabetes, gout, cardiovascular disorders, reperfusion injury, atherosclerosis, ischaemic heart disease, cardiac failure, stroke, liver disease, AIDS, AIDS related complex, neurological disorders, male infertility, ageing and infections, including plasmodium infection, bacterial infection and viral infection, particularly human herpesvirus 5 (cytomegalovirus) infection, even more particularly, hematological disease, leukocyte disorders including leukopenia, drug-induced leukopenia and leukemia, bone marrow transplantation, bone marrow disease, wound healing, immune disorders such as graft versus host disease and Crohn's disease, neoplastic disorders, melanoma, solid tumour, hyperlipidemia, hyperphosphatemia, anemia, ischemia, stroke, vascular disease, thrombosis, thromboembolism, infarction and infection, most particularly fungal and bacterial infection. In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be used in screening regimes for the identification of compounds that are effective in the treatment or diagnosis of such a disease.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D.N Glover ed. 1985); Oligonucleotide Synthesis (M.J. Gait ed. 1984); Nucleic Acid Hybridization (B.D. Hames & S.J. Higgins eds. 1984); Transcription and Translation (B.D. Hames & S.J. Higgins eds. 1984); Animal Cell Culture (R.I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J.H. Miller and M.P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D.M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro- protein that can be activated by cleavage of the pre-, pro- or prepro- portion to produce an active mature polypeptide. In such polypeptides, the pre-, pro- or prepro- sequence may be a leader or secretory sequence or may be a sequence that is employed for purification of the mature polypeptide sequence.

The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the first aspect of the invention may be polypeptides that are homologous to the INSP032, INSP033, INSP034, INSP036, or INSP038 exon polypeptides and/or to the INSP032, INSP033, INSP034, INSP036, or INSP038 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A.M., ed., Oxford University Press, N.Y., 1988; Biocomputing. Informatics and Genome Projects, Smith, D.W., ed., Academic Press, N.Y., 1993; Computer Analysis of Sequence Data, Part 1, Griffin, A.M., and Griffin, H.G., eds., Humana Press, N.J., 1994; Sequence Analysis in Molecular Biology, von Heinje, G., Academic Press, 1987; and Sequence Analysis Primer, Gribskov, M. and Devereux, J., eds., M Stockton Press, N.Y., 1991).

Homologous polypeptides therefore include natural biological variants (for example, allelic variants or geographical variations within the species from which the polypeptides are derived) and mutants (such as mutants containing amino acid substitutions, insertions or deletions) of the INSP032, INSP033, INSP034, INSP036 and INSP038 exon polypeptides and of the INSP032, INSP033, INSP034, INSP036 and INSP038 polypeptides. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions.

Such mutants also include polypeptides in which one or more of the amino acid residues includes a substituent group;

Typically, greater than 80% identity between two polypeptides is considered to be an indication of functional equivalence. Preferably, functionally equivalent polypeptides of the first aspect of the invention have a degree of sequence identity with the INSP032, INSP033, INSP034, INSP036 and INSP038 exon polypeptides or the INSP032, INSP033, INSP034, INSP036, or INSP038 polypeptides, or with active fragments thereof, of greater than 80%. More preferred polypeptides have degrees of identity of greater than 90%, 95%, 98% or 99%, respectively.

The functionally-equivalent polypeptides of the first aspect of the invention may also be polypeptides which have been identified using one or more techniques of structural alignment. For example, the Inpharmatica Genome Threader technology that forms one aspect of the search tools used to generate the Biopendium search database may be used (see co-pending PCT patent application PCT/GB01/01105) to identify polypeptides of presently-unknown function which, while having low sequence identity as compared to the INSP032, INSP034, INSP036 or INSP038 exon polypeptides or the INSP032, INSP034, INSP036, or INSP038 polypeptides, are predicted to have secreted molecule activity, by virtue of sharing significant structural homology with the INSP032, INSP034, INSP036 or INSP038 exon polypeptides or the INSP032, INSP034, INSP036, or INSP038 polypeptide sequences. By way of another example, the Inpharmatica Genome Threader technology that forms one aspect of the search tools used to generate the Biopendium search database may be used (see co-pending PCT patent application PCT/GB01/01105) to identify polypeptides of presently-unknown function which, while having low sequence identity as compared to the INSP033 exon polypeptide or the INSP033 polypeptides, are predicted to have granulocyte colony-stimulating factor, by virtue of sharing significant structural homology with the INSP033 exon polypeptide or the INSP033 polypeptides sequences. By “significant structural homology” is meant that the Inpharmatica Genome Threader predicts two proteins to share structural homology with a certainty of 10% and above.

The polypeptides of the first aspect of the invention also include fragments of the INSP032, INSP033, INSP034, INSP036, and INSP038 exon polypeptides and the INSP032, INSP033, INSP034, INSP036 and INSP038 polypeptides and fragments of the functional equivalents of these polypeptides, provided that those fragments retain secreted protein activity, in particular four helical bundle cytokine activity, and even more particularly, granulocyte colony stimulating factor activity or have an antigenic determinant in common with these polypeptides.

As used herein, the term “fragment” refers to a polypeptide having an amino acid sequence that is the same as part, but not all, of the amino acid sequence of the INSP032, INSP033, INSP034, INSP036 or INSP038 exon polypeptides or the INSP032, INSP033, INSP034, INSP036 or INSP038 polypeptides or one of its functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro- polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)2 and Fv, which are capable of binding to the antigenic determinant in question. Such antibodies thus bind to the polypeptides of the first aspect of the invention.

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl Acad. Sci. USA, 86, 10029 (1989); Gorman et al., Proc. Natl Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second and third aspects of the invention are those which encode the polypeptide sequences recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:24, SEQ ID NO:26, SEQ ID NO:28, SEQ ID NO:30, SEQ ID NO:32, SEQ ID NO:34, SEQ ID NO:38, SEQ ID NO:40, SEQ ID NO:42, SEQ ID NO:44, SEQ ID NO:46 and SEQ ID NO:48 and functionally equivalent polypeptides. These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones ,and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P.E. et al. (1993) Anticancer Drug Des. 8:53-63).

A nucleic acid molecule which encodes the polypeptide of SEQ ID NO:2 may be identical to the coding sequence of the nucleic acid molecule shown in SEQ ID NO:1. A nucleic acid molecule which encodes the polypeptide of SEQ ID NO:4 may be identical to the coding sequence of the nucleic acid molecule shown in SEQ ID NO:3 . A nucleic acid molecule which encodes the polypeptide of SEQ ID NO:6 may be identical to the coding sequence of the nucleic acid molecule shown in SEQ ID NO:5. A nucleic acid molecule which encodes the polypeptide of SEQ ID NO:8 may be identical to the coding sequence of the nucleic acid molecule shown in SEQ ID NO:7. A nucleic acid molecule which encodes the polypeptide of SEQ ID NO:10 may be identical to the coding sequence of the nucleic acid molecule shown in SEQ ID NO:9. A nucleic acid molecule which encodes the polypeptide of SEQ ID NO:12 may be identical to the coding sequence of the nucleic acid molecule shown in SEQ ID NO:11. A nucleic acid molecule which encodes the polypeptide of SEQ ID NO:14 may be identical to the coding sequence of the nucleic acid molecule shown in SEQ ID NO:13. A nucleic acid molecule which encodes the polypeptide of SEQ ID NO:16 may be identical to the coding sequence of the nucleic acid molecule shown in SEQ ID NO:15. A nucleic acid molecule which encodes the polypeptide of SEQ ID NO:24 may be identical to the coding sequence of the nucleic acid molecule shown in SEQ ID NO:23. A nucleic acid molecule which encodes the polypeptide of SEQ ID NO:26 may be identical to the coding sequence of the nucleic acid molecule shown in SEQ ID NO:25. A nucleic acid molecule which encodes the polypeptide of SEQ ID NO:28 may be identical to the coding sequence of the nucleic acid molecule shown in SEQ ID NO:27. A nucleic acid molecule which encodes the polypeptide of SEQ ID NO:30 may be identical to the coding sequence of the nucleic acid molecule shown in SEQ ID NO:29. A nucleic acid molecule which encodes the polypeptide of SEQ ID NO:32 may be identical to the coding sequence of the nucleic acid molecule shown in SEQ ID NO:31. A nucleic acid molecule which encodes the polypeptide of SEQ ID NO:34 may be identical to the coding sequence of the nucleic acid molecule shown in SEQ ID NO:33. A nucleic acid molecule which encodes the polypeptide of SEQ ID NO:38 may be identical to the coding sequence of the nucleic acid molecule shown in SEQ ID NO:37. A nucleic acid molecule which encodes the polypeptide of SEQ ID NO:40 may be identical to the coding sequence of the nucleic acid molecule shown in SEQ ID NO:39. A nucleic acid molecule which encodes the polypeptide of SEQ ID NO:42 may be identical to the coding sequence of the nucleic acid molecule shown in SEQ ID NO:41. A nucleic acid molecule which encodes the polypeptide of SEQ ID NO:44 may be identical to the coding sequence of the nucleic acid molecule shown in SEQ ID NO:43. A nucleic acid molecule which encodes the polypeptide of SEQ ID NO:46 may be identical to the coding sequence of the nucleic acid molecule shown in SEQ ID NO:45. A nucleic acid molecule which encodes the polypeptide of SEQ ID NO:48 may be identical to the coding sequence of the nucleic acid molecule shown in SEQ ID NO:47. These molecules also may have a different sequence which, as a result of the degeneracy of the genetic code, encodes a polypeptide of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:24, SEQ ID NO:26, SEQ ID NO:28, SEQ ID NO:30, SEQ ID NO:32, SEQ ID NO:34, SEQ ID NO:38, SEQ ID NO:40, SEQ ID NO:42, SEQ ID NO:44, SEQ ID NO:46 and SEQ ID NO:48. Such nucleic acid molecules may include, but are not limited to, the coding sequence for the mature polypeptide by itself; the coding sequence for the mature polypeptide and additional coding sequences, such as those encoding a leader or secretory sequence, such as a pro-, pre- or prepro- polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention. Such a nucleic acid molecule may be a naturally-occurring variant such as a naturally-occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or third aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J.S., Trends in Pharm. Sci., 10, 435 (1989), Okano, J. Neurochem. 56, 560 (1991); O° Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al [supra]). A substantially homologous molecule will then compete for and inhibit the binding of a completely homologous molecule to the target molecule under various conditions of stringency, as taught in Wahl, G.M. and S.L. Berger (1987; Methods Enzymol. 152:399-407) and Kimmel, A.R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5× Denhardts solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. (see Sambrook et al. [supra]). Preferably, the conditions used for hybridization are those of high stringency.

Preferred embodiments of this aspect of the invention are nucleic acid molecules that are at least 70% identical over their entire length to a nucleic acid molecule encoding the INSP032 polypeptide (SEQ ID NO:2 and SEQ ID NO:4 combined, equivalent to SEQ ID NO:6), INSP033 polypeptide (SEQ ID NO:8), INSP034 polypeptide (SEQ ID NO10, SEQ ID NO12 and SEQ ID NO:14 combined, equivalent to SEQ ID NO:16), INSP036 polypeptide (SEQ ID NO:24, SEQ ID NO:26, SEQ ID NO:28, SEQ ID NO:30 and SEQ ID NO:32 combined, equivalent to SEQ ID NO:34), and the INSP038 polypeptide (SEQ ID NO:38, SEQ ID NO:40, SEQ ID NO:42, SEQ ID NO:44 and SEQ ID NO:46 combined, equivalent to SEQ ID NO:48) and nucleic acid molecules that are substantially complementary to such nucleic acid molecules. Preferably, a nucleic acid molecule according to this aspect of the invention comprises a region that is at least 80% identical over its entire length to the nucleic acid molecules having the sequence produced by combining SEQ ID NO:1 and SEQ ID NO:3 (equivalent to SEQ ID NO:5) or SEQ ID NO:7 or SEQ ID NO:9, SEQ ID NO:11 and SEQ ID NO:13 (equivalent to SEQ ID NO:15) or SEQ ID NO:23, SEQ ID NO:25, SEQ ID NO:27, SEQ ID NO:29 and SEQ ID NO:31 (equivalent to SEQ ID NO:33) or SEQ ID NO:37, SEQ ID NO:39, SEQ ID NO:41, SEQ ID NO:43 and SEQ ID NO:45 (equivalent to SEQ ID NO:47) or a nucleic acid molecule that is complementary thereto. In this regard, nucleic acid molecules at least 90%, preferably at least 95%, more preferably at least 98% or 99% identical over their entire length to the same are particularly preferred. Preferred embodiments in this respect are nucleic acid molecules that encode polypeptides which retain substantially the same biological function or activity as the INSP032, INSP033, INSP034, INSP036 and INSP038 polypeptides.

Sequence identity with respect to any of the sequences presented here can be determined by a simple “eyeball” comparison (i.e. a strict comparison) of any one or more of the sequences with another sequence to see if that other sequence has, for example, at least 70% sequence identity to the sequence(s).

Alternatively, relative sequence identity can also be determined by commercially available computer programs that can calculate % identity between two or more sequences using any suitable algorithm for determining identity, using for example default parameters. A typical example of such a computer program is CLUSTAL. Other computer program methods to determine identify and similarity between the two sequences include but are not limited to the GCG program package (Devereux et al 1984 Nucleic Acids Research 12: 387) and FASTA (Atschul et al 1990 J Molec Biol 403-410).

The sequence identity or percent homology for proteins and nucleic acids can also be calculated as (Nref−Ndif)×100/Nref, wherein Ndif is the total number of non- identical residues in the two sequences when aligned and wherein Nref is the number of residues in one of the sequences. Hence, the DNA sequence AGTCAGTC will have a sequence identity of 75% with the sequence AATCAATC (Ndif=2 and Nref=8).

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (Ausubel et al., 1999 ibid-Chapter 18), FASTA (Atschul et al., 1990, J. Mol. Biol., 403-410) and the GENEWORKS suite of comparison tools. Both BLAST and FASTA are available for offline and online searching (Ausubel et al., 1999 ibid, pages 7-58 to 7-60).

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the INSP032, INSP033, INSP034, INSP036 and INSP038 polypeptides and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INSP032, INSP033, INSP034, INSP036 and INSP038 polypeptides is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989,1992). Probes comprising at least 15, preferably at least 30, and more preferably at least 50, contiguous bases that correspond to, or are complementary to, nucleic acid sequences from the appropriate encoding gene (SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:23, SEQ ID NO:25, SEQ ID NO:27, SEQ ID NO:29, SEQ ID NO:31, SEQ ID NO:33, SEQ ID NO:37, SEQ ID NO:39, SEQ ID NO:41, SEQ ID NO:43, SEQ ID NO:45 and SEQ ID NO:47) are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., PNAS USA 85, 8998-9002, 1988). Recent modifications of this technique, exemplified by the MarathonTM technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic., 1, 111-119). Another method which may be used to retrieve unknown sequences is that of Parker, J.D. et al. (1991); Nucleic Acids Res. 19:3055-3060). Additionally, one may use PCR, nested primers, and PromoterFinderTM libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes, using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

The vectors of the present invention comprise nucleic acid molecules of the invention and may be cloning or expression vectors. The host cells of the invention, which may be transformed, transfested or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al.,[supra]. Particularly suitable methods include calcium phosphate transfection, DEAE-dextran mediated transfection, transvection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSport1TM plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture has been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk− or aprt± cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D.E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio)).

Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well- known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well-known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992), Prot. Exp. Purif. 3: 263-281) while the thioredoxin or enterokinase cleavage site provides a means for purifying the polypeptide from the fusion protein. A discussion of vectors which contain fusion proteins is provided in Kroll, D.J. et al. (1993; DNA Cell Biol. 12:441-453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

The polypeptides of the present invention may modulate a variety of physiological and pathological processes. Thus, the biological activity of these polypeptides can be examined in systems that allow the study of such modulatory activities, using a variety of suitable assays.

For example, such assays may include, but are not limited to, those assays documented in Nagata S & Fukunaga, R Granulocyte colony-stimulating factor and its receptor, Prog Growth Factor Res. 1991;3(2):131-41. Review; Wells JA. Binding in the growth hormone receptor complex, Proc Natl Acad Sci USA. 1996 Jan. 9;93(1):1-6. Review; Hudson K R et al., Characterization of the receptor binding sites of human leukemia inhibitory factor and creation of antagonists. J Biol Chem. 1996 May 17;271(20):11971-8; Park H et al., Identification of functionally important residues of human thrombopoietin. J Biol Chem. 1998 Jan. 2;273(l):256-61; Panayotatos N, et al, Localization of functional receptor epitopes on the structure of ciliary neurotrophic factor indicates a conserved, function-related epitope topography among helical cytokines. J Biol Chem. 1995 Jun. 9;270(23):14007-14; Lu CM, et al., Increasing bioactivity of flt3 ligand by fusing two identical soluble domains, Sheng Wu Hua Xue Yu Sheng Wu Wu Li Xue Bao (Shanghai), November 2002;34(6):697-702; Vigano P, et al., Expression of interleukin-10 and its receptor is up-regulated in early pregnant versus cycling human endometrium J Clin Endocrinol Metab. December 2002; 87(12):5730-6 for determining 4-helical bundle cytokines function and therefore function of INSP033. In addition, granulocyte colony stimulating factor is one of the hematopoietic growth factors that regulates the proliferation and differentiation of bone marrow progenitor cell populations. Activity can therefore be evaluated by measuring bone marrow progenitor cell proliferation.

A preferred method for identifying an agonist or antagonist compound of a polypeptide of the present invention comprises:
(a) contacting a cell expressing on the surface thereof the polypeptide according to the first aspect of the invention, the polypeptide being associated with a second component capable of providing a detectable signal in response to the binding of a compound to the polypeptide, with a compound to be screened under conditions to permit binding to the polypeptide; and
(b) determining whether the compound binds to and activates or inhibits the polypeptide by measuring the level of a signal generated from the interaction of the compound with the polypeptide.

A further preferred method for identifying an agonist or antagonist of a polypeptide of the invention comprises:
(a) contacting a cell expressing on the surface thereof the polypeptide, the polypeptide being associated with a second component capable of providing a detectable signal in response to the binding of a compound to the polypeptide, with a compound to be screened under conditions to permit binding to the polypeptide; and
(b) determining whether the compound binds to and activates or inhibits the polypeptide by comparing the level of a signal generated from the interaction of the compound with the polypeptide with the level of a signal in the absence of the compound.

In further preferred embodiments, the general methods that are described above may further comprise conducting the identification of agonist or antagonist in the presence of labelled or unlabelled ligand for the polypeptide.

In another embodiment of the method for identifying agonist or antagonist of a polypeptide of the present invention comprises:
determining the inhibition of binding of a ligand to cells which have a polypeptide of the invention on the surface thereof, or to cell membranes containing such a polypeptide, in the presence of a candidate compound under conditions to permit binding to the polypeptide, and determining the amount of ligand bound to the polypeptide. A compound capable of causing reduction of binding of a ligand is considered to be an agonist or antagonist. Preferably the ligand is labelled.

More particularly, a method of screening for a polypeptide antagonist or agonist compound comprises the steps of:
(a) incubating a labelled ligand with a whole cell expressing a polypeptide according to the invention on the cell surface, or a cell membrane containing a polypeptide of the invention,
(b) measuring the amount of labelled ligand bound to the whole cell or the cell membrane;
(c) adding a candidate compound to a mixture of labelled ligand and the whole cell or the cell membrane of step (a) and allowing the mixture to attain equilibrium;
(d) measuring the amount of labelled ligand bound to the whole cell or the cell membrane after step (c); and
(e) comparing the difference in the labelled ligand bound in step (b) and (d), such that the compound which causes the reduction in binding in step (d) is considered to be an agonist or antagonist.

The polypeptides may be found to modulate a variety of physiological and pathological processes in a dose-dependent manner in the above-described assays. Thus, the “functional equivalents” of the polypeptides of the invention include polypeptides that exhibit any of the same modulatory activities in the above-described assays in a dose-dependent manner. Although the degree of dose-dependent activity need not be identical to that of the polypeptides of the invention, preferably the “functional equivalents” will exhibit substantially similar dose-dependence in a given activity assay compared to the polypeptides of the invention.

In certain of the embodiments described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Assay methods that are also included within the terms of the present invention are those that involve the use of the genes and polypeptides of the invention in overexpression or ablation assays. Such assays involve the manipulation of levels of these genes/polypeptides in cells and assessment of the impact of this manipulation event on the physiology of the manipulated cells. For example, such experiments reveal details of signaling and metabolic pathways in which the particular genes/polypeptides are implicated, generate information regarding the identities of polypeptides with which the studied polypeptides interact and provide clues as to methods by which related genes and proteins are regulated.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X] is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intraarterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion.

Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J.E. et al. (1994) In: Huber, B.E. and B.I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K.L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (ie. to prevent infection) or therapeutic (ie. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al., Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:
a) contacting a sample of tissue from the patient with a nucleic acid probe under stringent conditions that allow the formation of a hybrid complex between a nucleic acid molecule of the invention and the probe;
b) contacting a control sample with said probe under the same conditions used in step a);
c) and detecting the presence of hybrid complexes in said samples;
wherein detection of levels of the hybrid complex in the patient sample that differ from levels of the hybrid complex in the control sample is indicative of disease.

A further aspect of the invention comprises a diagnostic method comprising the steps of:
a) obtaining a tissue sample from a patient being tested for disease;
b) isolating a nucleic acid molecule according to the invention from said tissue sample; and
c) diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250, 559-562 (1990), and Trask et al., Trends, Genet., 7, 149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M.Chee et al., Science (1996), Vol 274, pp 610-613).

In one embodiment, the array is prepared and used according to the methods described in PCT application WO95/11995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or 10 any other suitable solid support. In another aspect, an oligonucleotide may be synthesized on the surface of the substrate by using a chemical coupling procedure and an ink jet application apparatus, as described in PCT application WO95/251116 (Baldeschweiler et al). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:
(a) a nucleic acid molecule of the present invention;
(b) a polypeptide of the present invention; or
(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease, particularly immune disorders, such as autoimmune disease, rheumatoid arthritis, osteoarthritis, psoriasis, systemic lupus erythematosus, and multiple sclerosis, inflammatory disorders, such as allergy, rhinitis, conjunctivitis, glomerulonephritis, uveitis, Crohn's disease, ulcerative colitis, inflammatory bowel disease, pancreatitis, digestive system inflammation, sepsis, endotoxic shock, septic shock, cachexia, myalgia, ankylosing spondylitis, myasthenia gravis, post-viral fatigue syndrome, pulmonary disease, respiratory distress syndrome, asthma, chronic-obstructive pulmonary disease, airway inflammation, wound healing, endometriosis, dermatological disease, Behcet's disease, neoplastic disorders, such as melanoma, sarcoma, renal tumour, colon tumour, haematological disease, myeloproliferative disorder, Hodgkin's disease, osteoporosis, obesity, diabetes, gout, cardiovascular disorders, reperfusion injury, atherosclerosis, ischaemic heart disease, cardiac failure, stroke, liver disease, AIDS, AIDS related complex, neurological disorders, male infertility, ageing and infections, including plasmodium infection, bacterial infection and viral infection, particularly human herpesvirus 5 (cytomegalovirus) infection. Even more preferably, such diseases include hematological disease, leukocyte disorders including leukopenia, drug-induced leukopenia and leukemia, bone marrow transplantation, bone marrow disease, wound healing, immune disorders such as graft versus host disease and Crohn's disease, neoplastic disorders, melanoma, solid tumour, hyperlipidemia, hyperphosphatemia, anemia, ischemia, stroke, vascular disease, thrombosis, thromboembolism, infarction and infection, most particularly fungal and bacterial infection.

Various aspects and embodiments of the present invention will now be described in more detail by way of example, with particular reference to INSP032, INSP033, INSP034, INSP036 and INSP038 polypeptides.

It will be appreciated that modification of detail may be made without departing from the scope of the invention.

### BRIEF DESCRIPTION OF THE FIGURES

FIG. 1: Results from Inpharmatica Genome Threader query using combined SEQ ID NO:2 and SEQ ID NO:4 polypeptide sequences (equivalent to SEQ ID NO:6).

FIG. 2: Alignment generated by Inpharmatica Genome Threader between combined SEQ ID NO:2 and SEQ ID NO:4 polypeptide sequence (equivalent to SEQ ID NO:6) and closest related structure.

FIG. 3: Results from Inpharmatica Genome Threader query SEQ ID NO:8a.

FIG. 4: Alignment generated by Inpharmatica Genome Threader between SEQ ID NO:8a and closest related structure.

FIG. 5: INSP033 predicted nucleotide sequence (SEQ ID NO:7a) with translation (SEQ ID NO:8a).

FIG. 6: INSP033 cloned nucleotide sequence (SEQ ID NO:7) with translation (SEQ ID NO:8).

FIG. 7: Pairwise alignment of cloned INSP033 nucleotide sequence (SEQ ID NO:7) against the predicted sequence (SEQ ID NO:7a).

FIG. 8: Pairwise alignment of amino acid sequence of cloned INSP033 (SEQ ID NO:8) compared to the predicted sequence (SEQ ID NO:8a).

FIG. 9: Map of PCRII-TOPO-IPAAA24020.

FIG. 10: Map of plasmid pDONR201.

FIG. 11: Map of expression vector pEAK12d.

FIG. 12: Plasmid map of pEAK12d-IPAAA24020.

FIG. 13: Nucleotide sequence of PCRII-TOPO-IPAAA24020.

FIG. 14: Nucleotide sequence of pEAK12D-IPAAA24020-6HIS.

FIG. 15: The NCBI-NR results for INSP033 polypeptide (SEQ ID NO:8) showing no 100% match, thus demonstrating INSP033 to be novel.

FIG. 16: The NCBI-month-aa results for INSP033 polypeptide (SEQ ID NO:8) showing no 100% match, thus demonstrating INSP033 to be novel.

FIG. 17: The NCBI-month-nt results for INSP033 nucleotide (SEQ ID NO:7) showing no 100% match, thus demonstrating INSP033 to be novel. The database used here is a translated nucleotide database, thus the results are represented in amino acids rather than nucleotides.

FIG. 18: Results from Inpharmatica Genome Threader query using combined SEQ ID NO:10, SEQ ID NO:12 and SEQ ID NO:14 polypeptide sequences (equivalent to SEQ ID NO:16).

FIG. 19: Alignment generated by Inpharmatica Genome Threader between combined SEQ ID NO:10, SEQ ID NO:12 and SEQ ID NO:14 polypeptide sequences (equivalent to SEQ ID NO:16) and closest related structure.

FIG. 20: Results from Inpharmatica Genome Threader query using combined SEQ ID NO:24 SEQ ID NO:26, SEQ ID NO:28, SEQ ID NO:30 and SEQ ID NO:32 polypeptide sequences (equivalent to SEQ ID NO:34).

FIG. 21: Alignment generated by Inpharmatica Genome Threader between combined SEQ ID NO:24 SEQ ID NO:26, SEQ ID NO:28, SEQ ID NO:30 and SEQ ID NO:32 polypeptide sequences (equivalent to SEQ ID NO:34) and closest related structure.

FIG. 22: Results from Inpharmatica Genome Threader query using combined SEQ ID NO:38 SEQ ID NO:40, SEQ ID NO:42, SEQ ID NO:44 and SEQ ID NO:46 polypeptide sequences (equivalent to SEQ ID NO:48).

FIG. 23: Alignment generated by Inpharmatica Genome Threader between combined SEQ ID NO:38 SEQ ID NO:40, SEQ ID NO:42, SEQ ID NO:44 and SEQ ID NO:46 polypeptide sequences (equivalent to SEQ ID NO:48) and closest related structure.

### EXAMPLES

### Example 1 INSP032

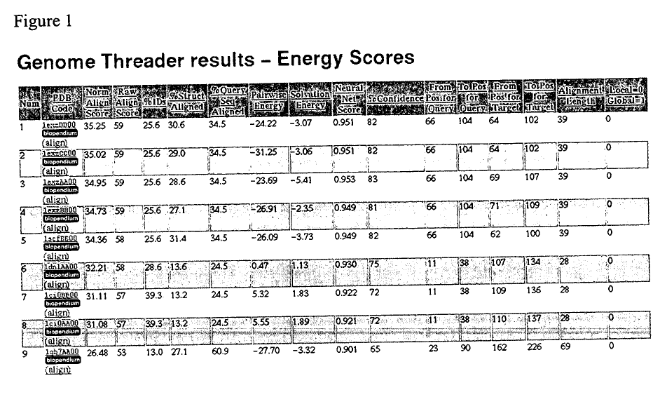

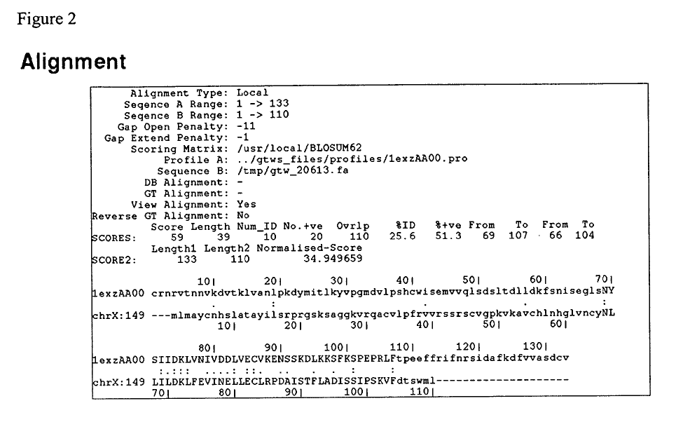

The polypeptide sequence derived from combining SEQ ID NO:2 and SEQ ID NO:4 (equivalent to SEQ ID NO:6) which represent the translation of consecutive exons from INSP032 were used as a query in the Inpharmatica Genome Threader tool against protein structures present in the PDB database. The top matches include structures of four helical bundle cytokine family members, the top five of which align to the query sequence with a Genome Threader confidence >80% (FIG. 1). FIG. 2 shows the alignment of the INSP032 query sequence to the sequence of stem cell factor (PDB-lexz) a member of the four helical bundle cytokine family (Zhang et al, Proc Natl Acad Sci USA. 2000 Jul. 5;97(14):7732-7). Note that the INSP032 polypeptide sequence is referred to as “chrX:149” in FIG. 2. Members of the four helical bundle cytokine family of secreted proteins are of therapeutic importance.

### Example 2 INSP033

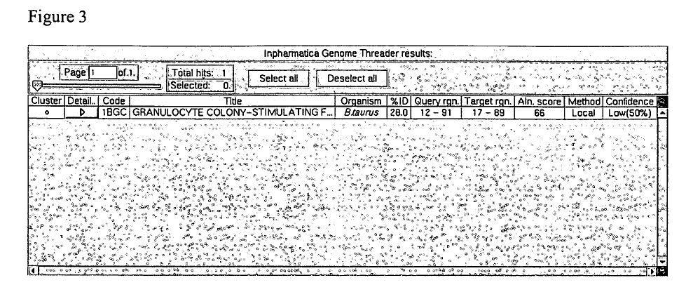

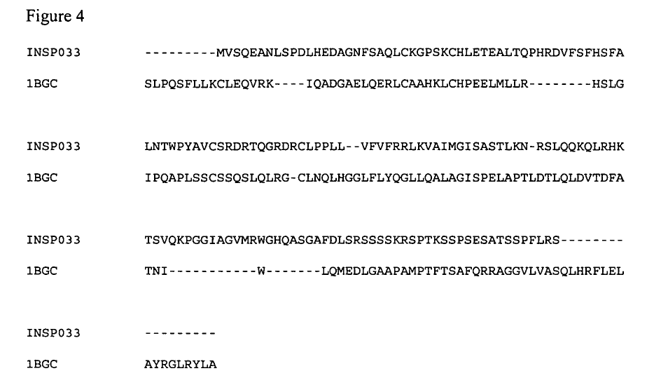

The polypeptide sequence derived from SEQ ID NO:8a which represents the translation of exons from INSP033 was used as a query in the Inpharmatica Genome Threader tool against protein structures present in the PDB database. The top match is the structure of a four helical bundle cytokine family member. The top match aligns to the query sequence with a Genome Threader confidence of 50% (FIG. 3). FIG. 4 shows the alignment of the INSP033 query sequence to the sequence of Granulocyte colony-stimulating factor (PDB-1bgc) a member of the four helical bundle cytokine family (Lovejoy et al, J Mol Biol. 1993 Dec. 5;234(3):640-53). Members of the four helical bundle cytokine family of proteins, more specifically, members of the granulocyte colony-stimulating factor family are of therapeutic importance.

The results of more experiments pertaining to INSP033 are shown below. INSP033 is also referred to as “IPAAA24020”.

1. Cloning of IPAAA24020 from cDNA Libraries

1.1 cDNA Libraries

Human cDNA libraries (in bacteriophage lambda (λ) vectors) were purchased from Stratagene or Clontech or prepared at the Serono Pharmaceutical Research Institute in λ ZAP or λ GT10 vectors according to the manufacturer's protocol (Stratagene). Bacteriophage λ DNA was prepared from small scale cultures of infected E.coli host strain using the Wizard Lambda Preps DNA purification system according to the manufacturer's instructions (Promega, Corporation, Madison Wisc.) The list of libraries and host strains used is shown in Table I.

1.2 PCR of Virtual cDNAs from Phage Library DNA

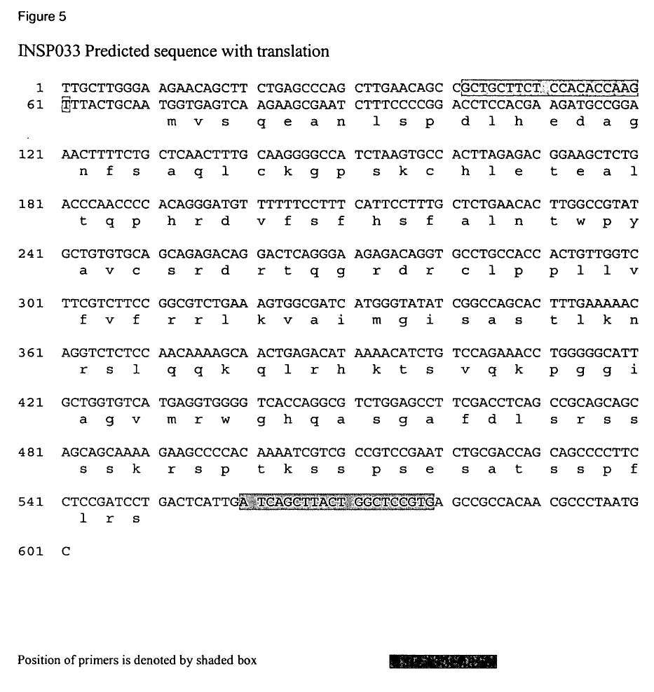

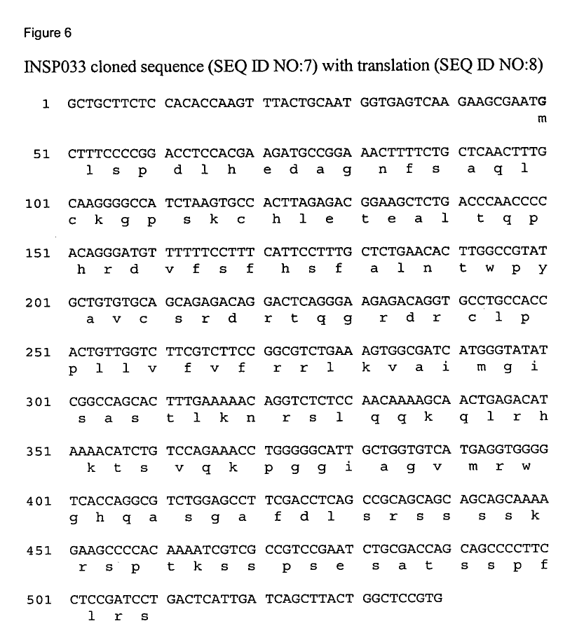

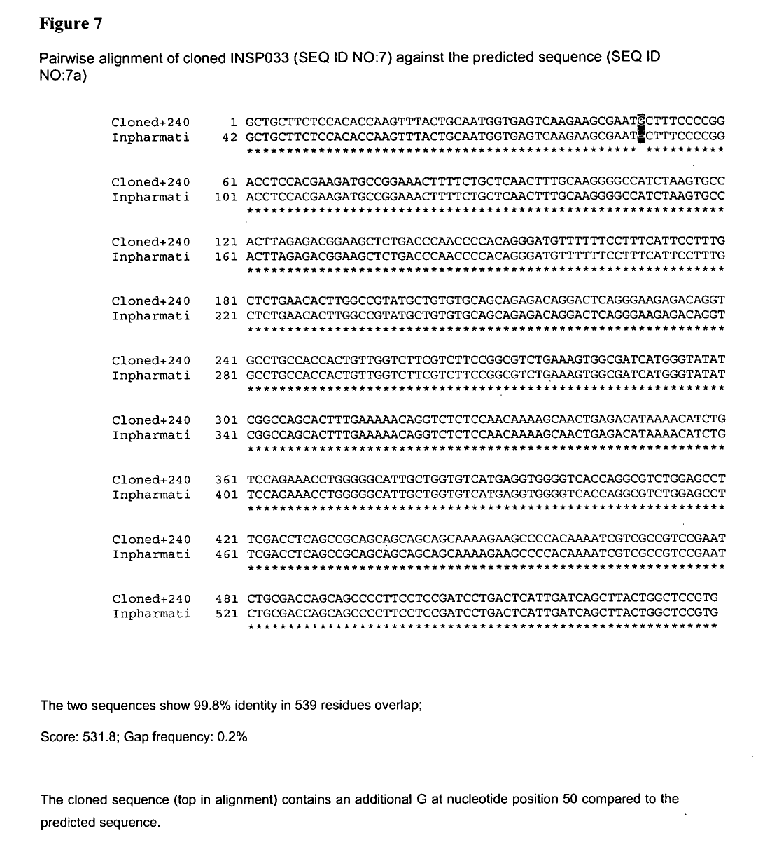

Full length virtual cDNA encoding IPAAA24020 (FIG. 5) was obtained as a PCR amplification product of 538 bp (FIG. 6) using gene specific cloning primers (CP1 and CP2, FIGS. 5 and 7). The PCR was performed in a final volume of 50 μl containing 1× AmpliTaq™ buffer, 200 μM dNTPs, 50 pmoles each of cloning primers primers, 2.5 units of AmpliTaq™ (Perkin Elmer) and 100 ng of each phage library DNA using an MJ Research DNA Engine, programmed as follows: 94° C., 1 min; 40 cycles of 94° C., 1 min, x ° C., and y min and 72° C., (where x is the lowest Tm−5° C. and y=1 min per kb of product); followed by 1 cycle at 72° C. for 7 min and a holding cycle at 4° C.

The amplification products were visualized on 0.8% agarose gels in 1×TAE buffer (Life Technologies) and PCR products migrating at the predicted molecular mass were purified from the gel using the Wizard PCR Preps DNA Purification System (Promega). PCR products eluted in 50 μl of sterile water were either subcloned directly or stored at −20° C.

1.3 Gene Specific Cloning Primers for PCR

Pairs of PCR primers having a length of between 18 and 25 bases were designed for amplifying the full length sequence of the virtual cDNA using Primer Designer Software (Scientific & Educational Software, PO Box 72045, Durham, N.C. 27722-2045, USA). PCR primers were optimized to have a Tm close to 55±10° C. and a GC content of 40-60%. Primers were selected which had high selectivity for the target sequence IPAAA24020 (little or no none specific priming to other templates).

1.4 Subcloning of PCR Products

PCR products were subcloned into the topoisomerase I modified cloning vector (pCR II TOPO) using the TOPO TA cloning kit purchased from the Invitrogen Corporation (cat. No. K4600-01 and K4575-01 respectively) using the conditions specified by the manufacturer. Briefly, 4 μl of gel purified PCR product from the human fetal kidney library (library number 12) amplification was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm SOC media (room temperature) was added. Samples were incubated with shaking (220 rpm) for 1 h at 37° C. The transformation mixture was then plated on L-broth (LB) plates containing ampicillin (100 μg/ml) and incubated overnight at 37° C. Ampicillin resistant colonies containing cDNA inserts were identified by colony PCR.

1.5 Colony PCR

Colonies were inoculated into 50 μl sterile water using a sterile toothpick. A 10 μl aliquot of the inoculum was then subjected to PCR in a total reaction volume of 20 μl as described above, except the primers pairs used were SP6 and T7. The cycling conditions were as follows: 94° C., 2 min; 30 cycles of 94° C., 30 sec, 47° C., 30 sec and 72° C. for 1 min); 1 cycle, 72° C., 7 min. Samples were then maintained at 4° C. (holding cycle) before further analysis.

PCR reaction products were analyzed on 1% agarose gels in 1×TAE buffer. Colonies which gave the expected PCR product size (538 bp cDNA+187 bp due to the multiple cloning site or MCS) were grown up overnight at 37° C. in 5 ml L-Broth (LB) containing ampicillin (50 μg/ml), with shaking at 220 rpm at 37° C.

1.6 Plasmid DNA Preparation and Sequencing

Miniprep plasmid DNA was prepared from 5 ml cultures using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer. Plasmid DNA (200-500 ng) was subjected to DNA sequencing with T7 primer and SP6 primer using the BigDyeTerminator system (Applied Biosystems cat. no. 4390246) according to the manufacturer's instructions. Sequencing reactions were purified using Dye-Ex columns (Qiagen) or Montage SEQ 96 cleanup plates (Millipore cat. no. LSKS09624) then analysed on an Applied Biosystems 3700 sequencer.

2. Construction of Plasmids for Expression of IPAAA24020 in HEK293/EBNA Cells.

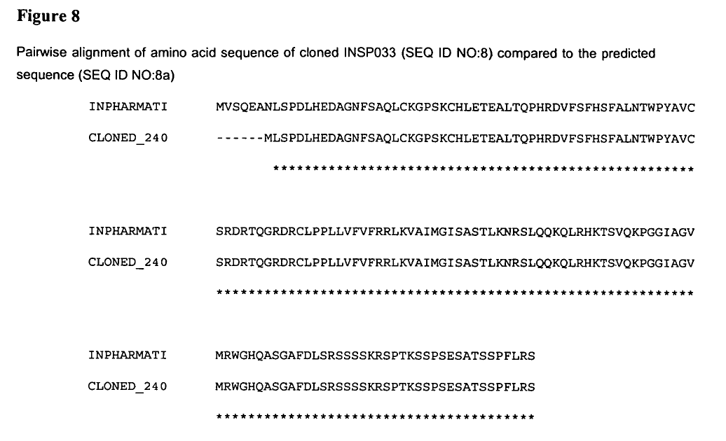

A clone containing the full coding sequence (ORF) of IPAAA24020 identified by DNA sequencing was then used to subclone the insert into the mammalian cell expression vector pEAK12d using the Gateway™ cloning methodology (Invitrogen). The cloned sequence of IPAAA24020 contains a single nucleotide G insertion at position 50 (FIG. 6) compared to the predicted sequence (FIG. 7). This insertion results in the generation of a new start codon at position 48 in the cloned sequence which is likely to be the translational start site as there is an upstream stop codon starting at nucleotide 33 in the cloned sequence. The new ORF has an identical sequence to the predicted amino sequence of EPAAA24020 except that it lacks the first 6 amino acids and amino acid 7 (Asn) in the virtual sequence is encodes the initiating methionine in the cloned sequence (FIG. 8).

2.1 Generation of Gateway Compatible IPAAA24020 ORF Fused to an in Frame 6HIS Tag Sequence.

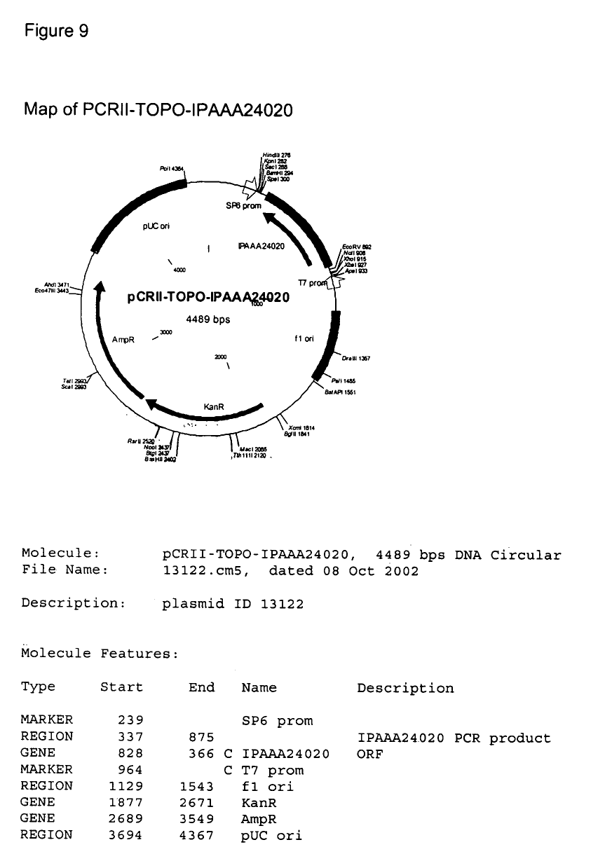

The first stage of the Gateway cloning process involves a two step PCR reaction which generates the ORF of IPAAA24020 flanked at the 5′ end by an attBl recombination site and Kozak sequence; and flanked at the 3′ end by a sequence encoding an in-frame 6 histidine (6HIS) tag, a stop codon and the attB2 recombination site (Gateway compatible cDNA). The first PCR reaction (in a final volume of 50 μl) contains: 25 ng of pCR II TOPO-IPAAA24022 (plasmid 13122 and FIG. 9), 2 μl dNTPs (5mM), 5 μl of 10×Pfx polymerase buffer, 0.5 μl each of gene specific primer (100 μM) (EX1 forward and EX1 reverse) and 0.5 μl Platinum Pfx DNA polymerase (Invitrogen). The PCR reaction was performed using an initial denaturing step of 95° C. for 2 min, followed by 12 cycles of 94° C., 15 sec and 68° C. for 30 sec. PCR products were purified directly from the reaction mixture using the Wizard PCR prep DNA purification system (Promega) according to the manufacturer's instructions. The second PCR reaction (in a final volume of 50 μl) contained 10 μl purified PCR product, 2 μl dNTPs (5 mM), 5 μl of 10×Pfx polymerase buffer, 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 45° C., 30 sec and 68° C. for 3.5 min; 25 cycles of 94° C., 15 sec; 55° C., 30 sec and 68° C., 3.5 min. PCR products were purified as described above.

2.2 Subcloning of Gateway Compatible IPAAA24020 ORF into Gateway Entry Vector pDONR201 and Expression Vector pEAK12d

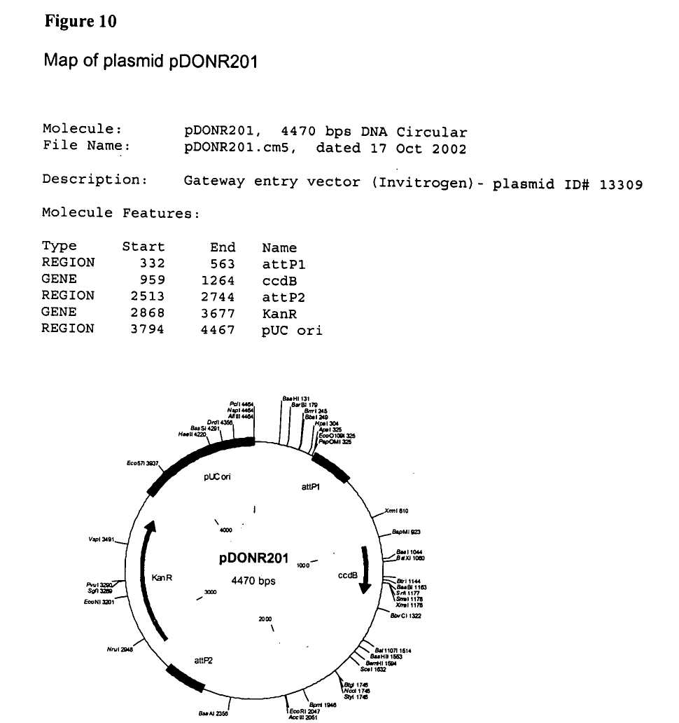

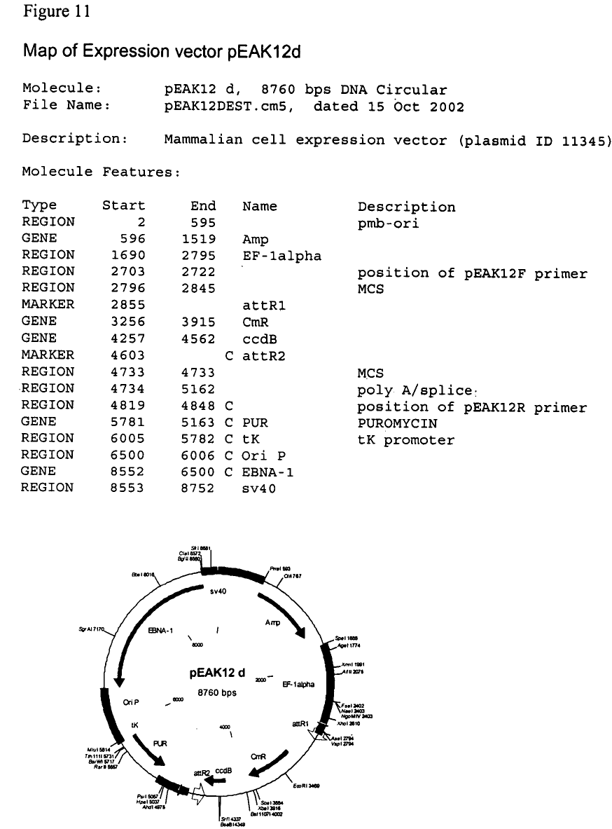

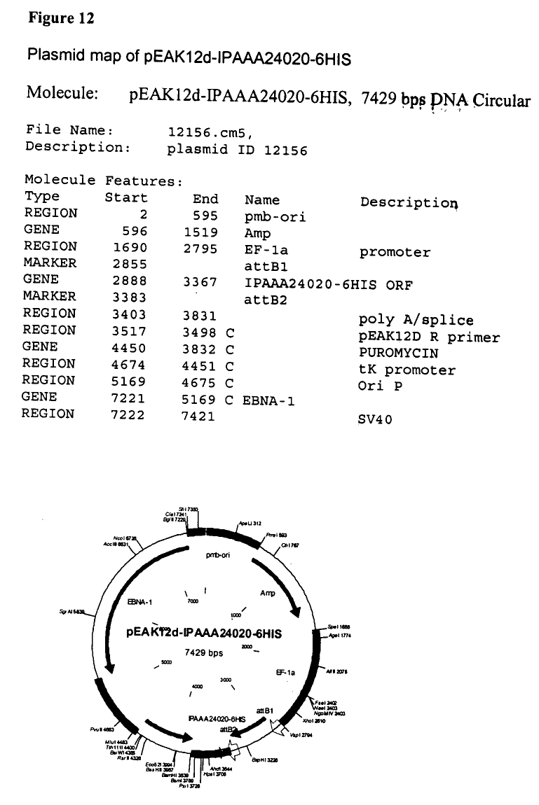

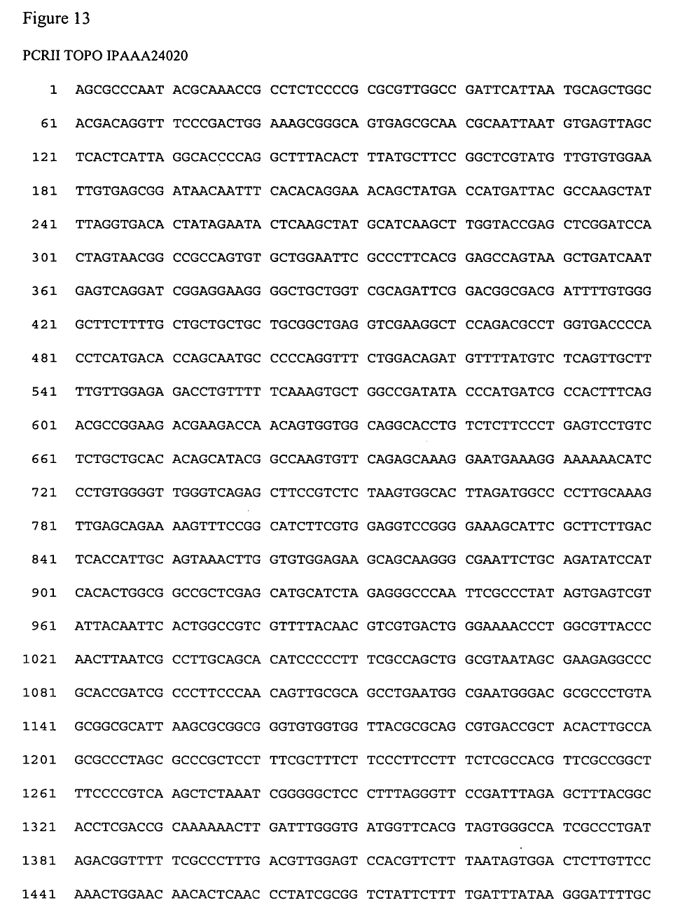

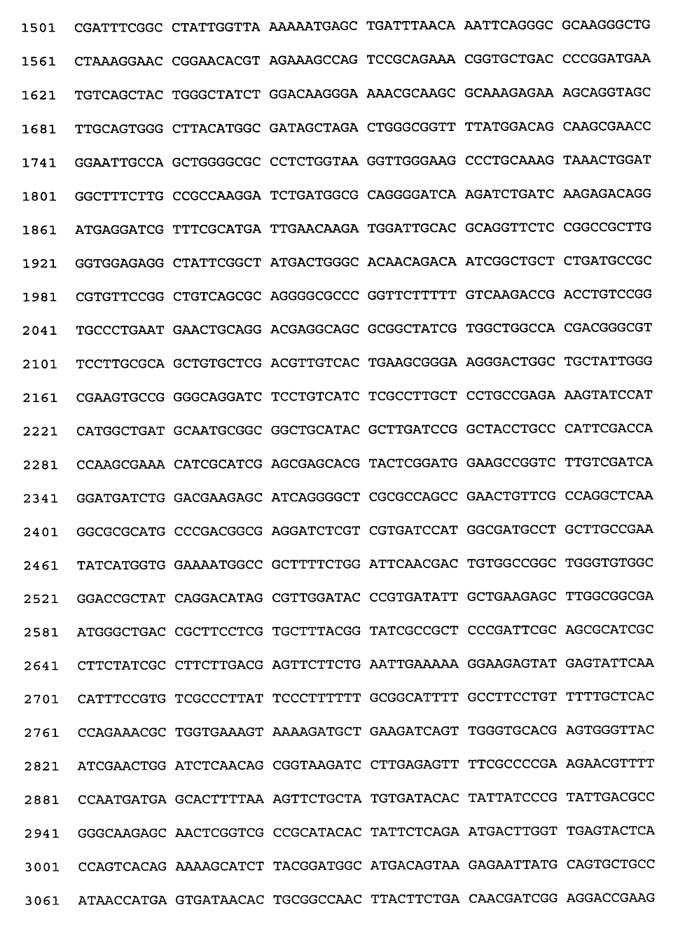

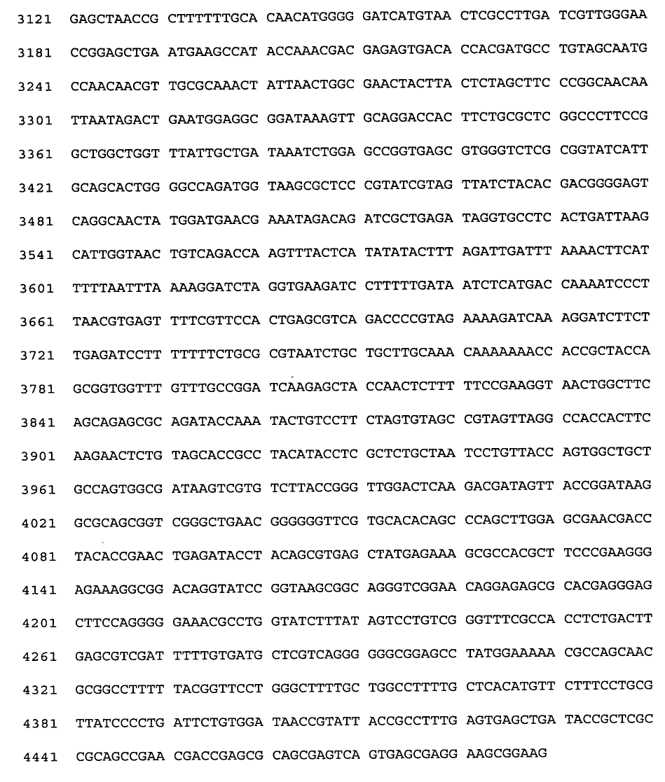

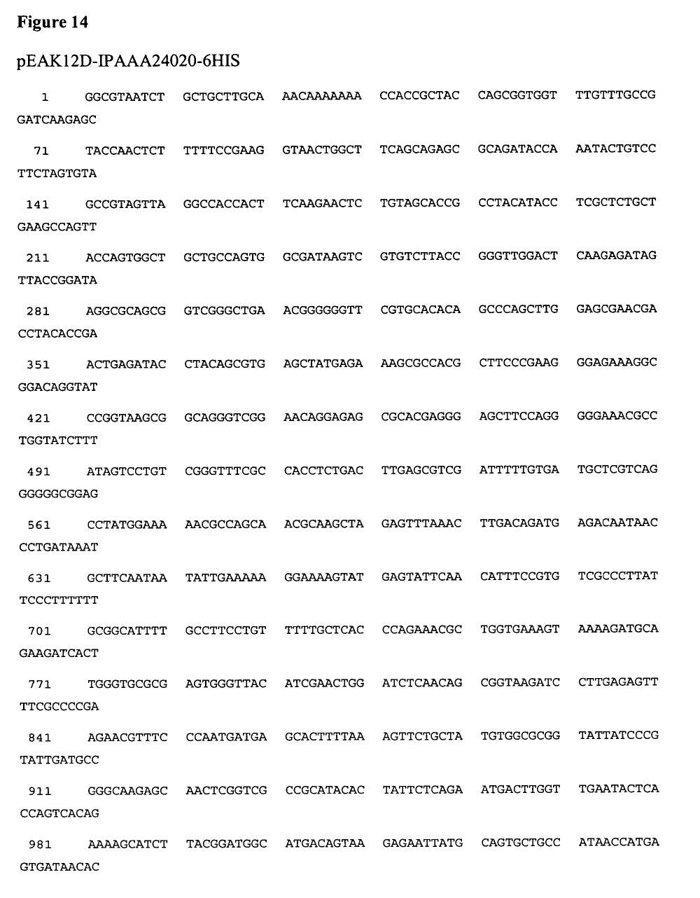

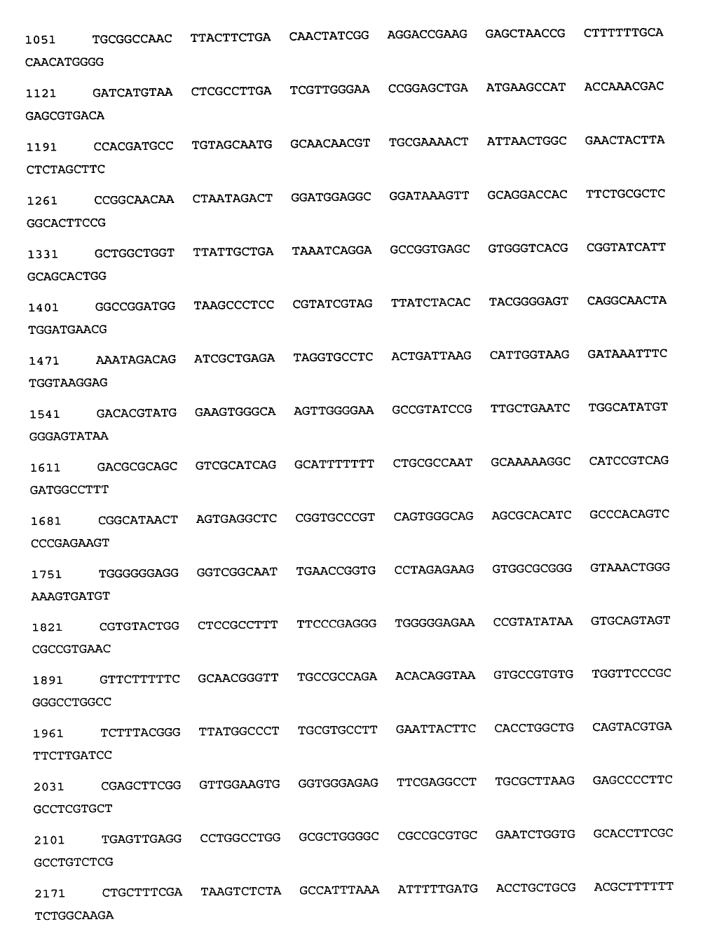

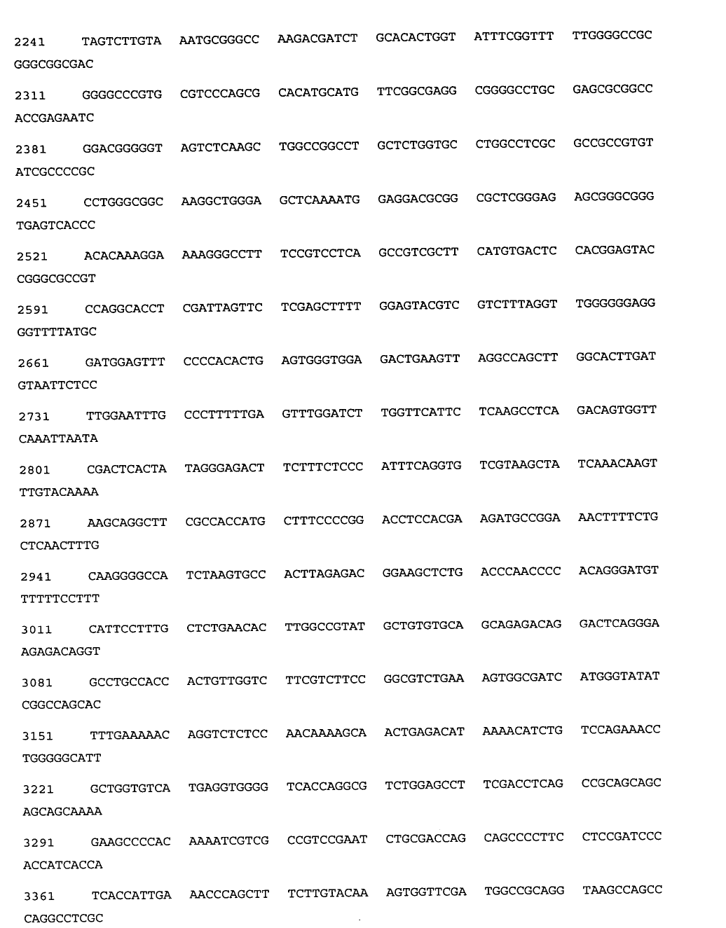

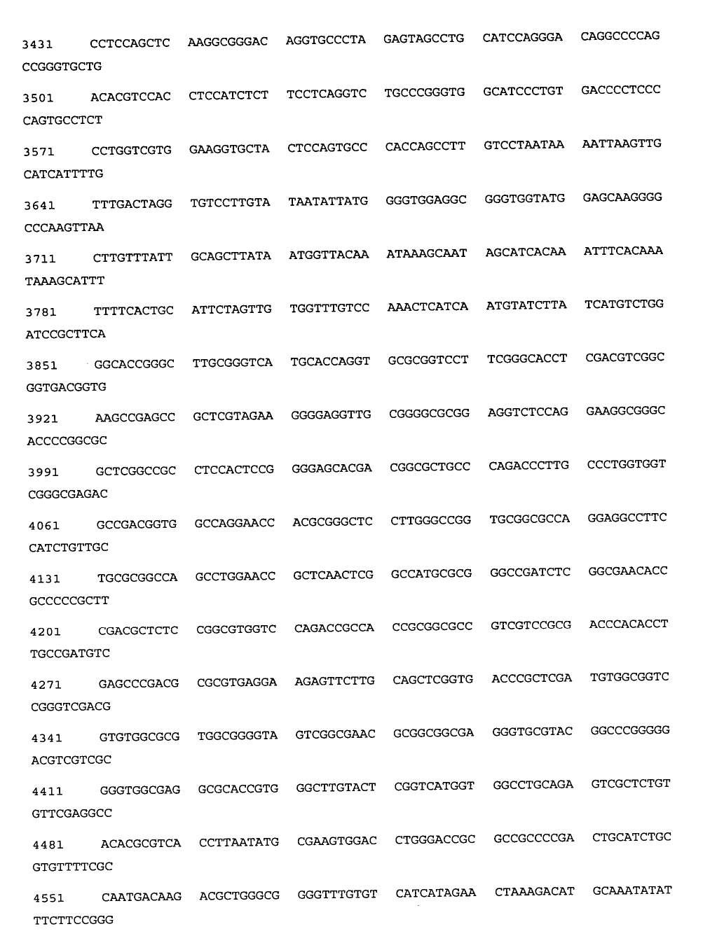

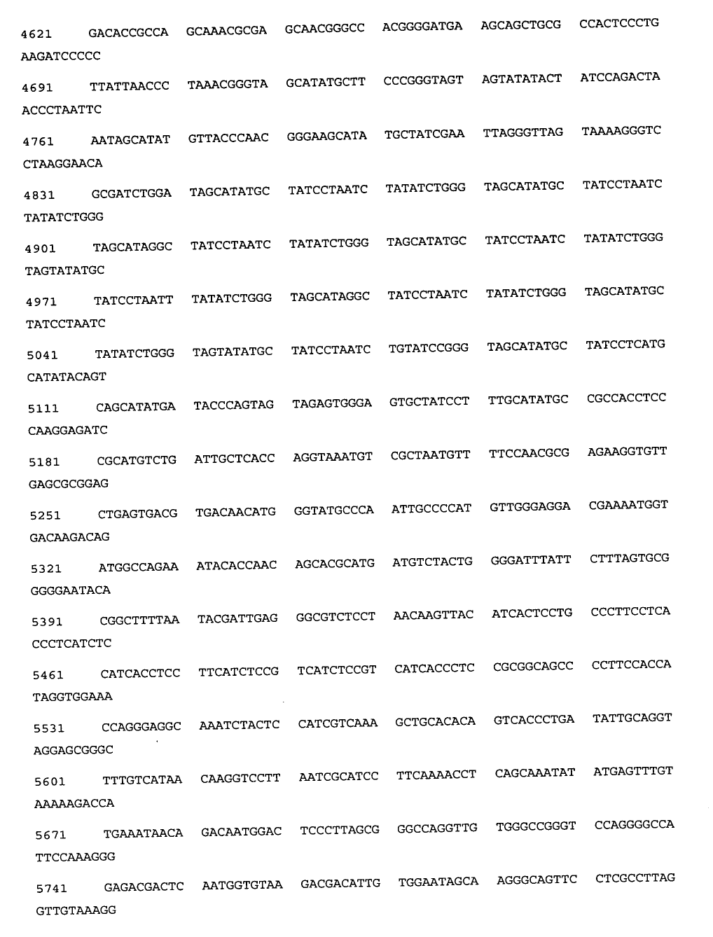

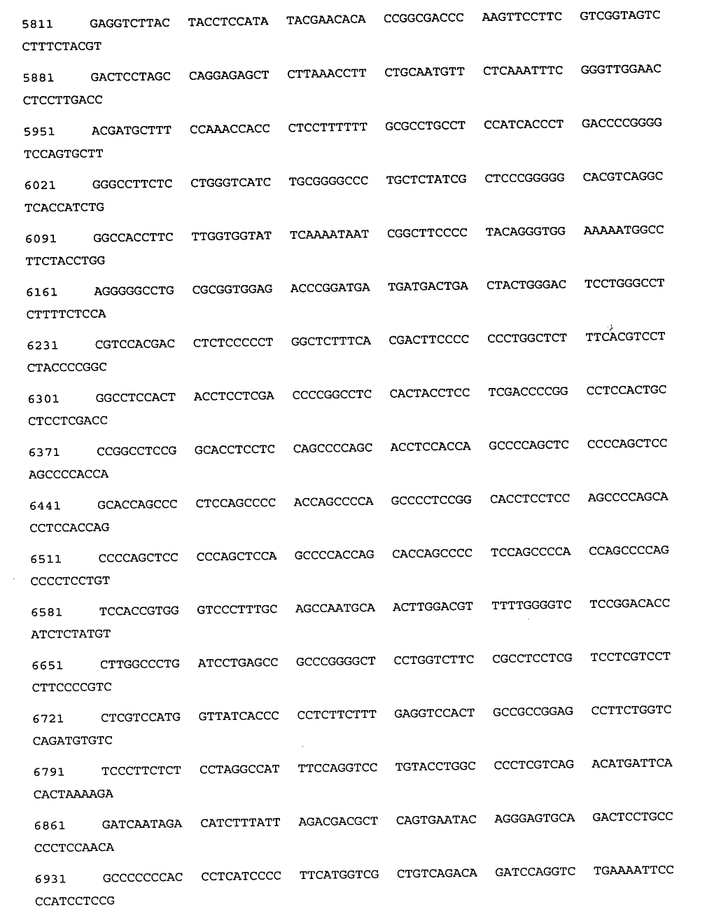

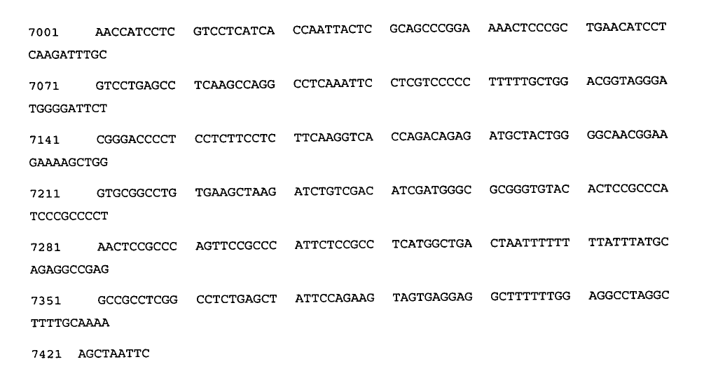

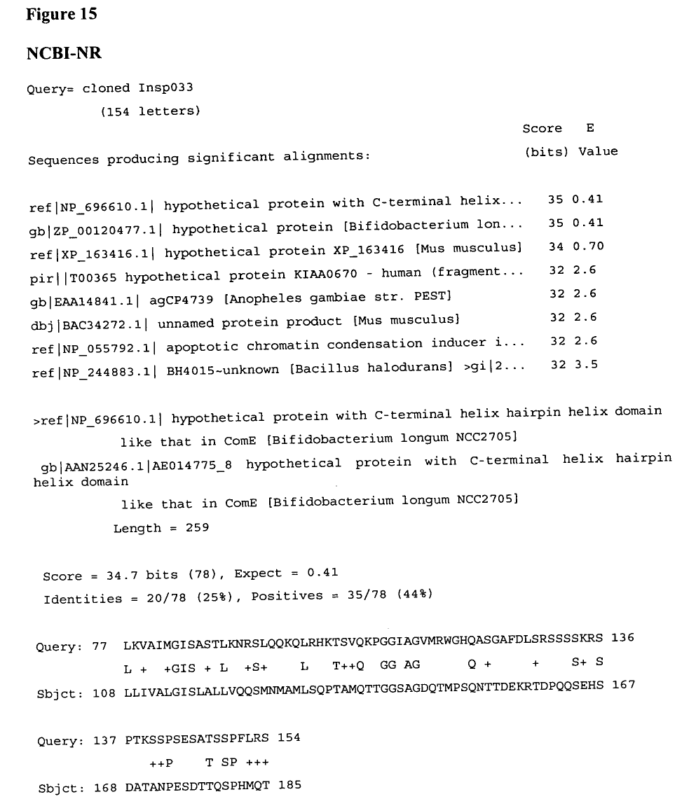

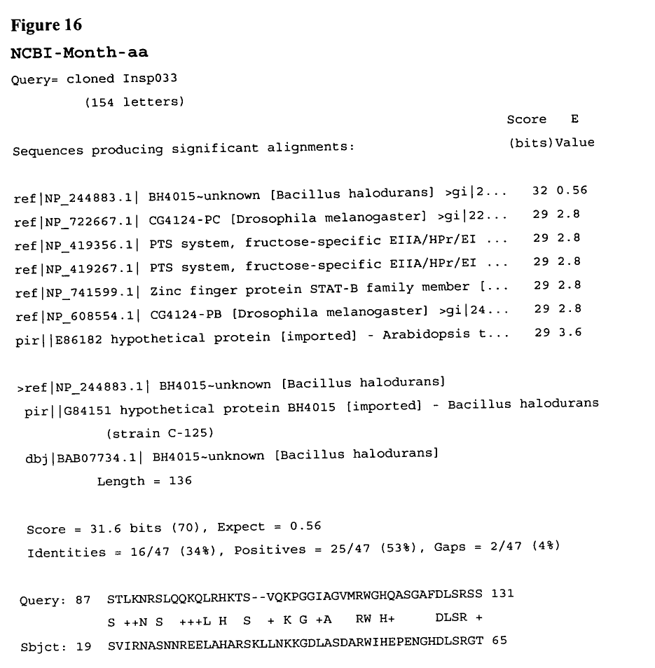

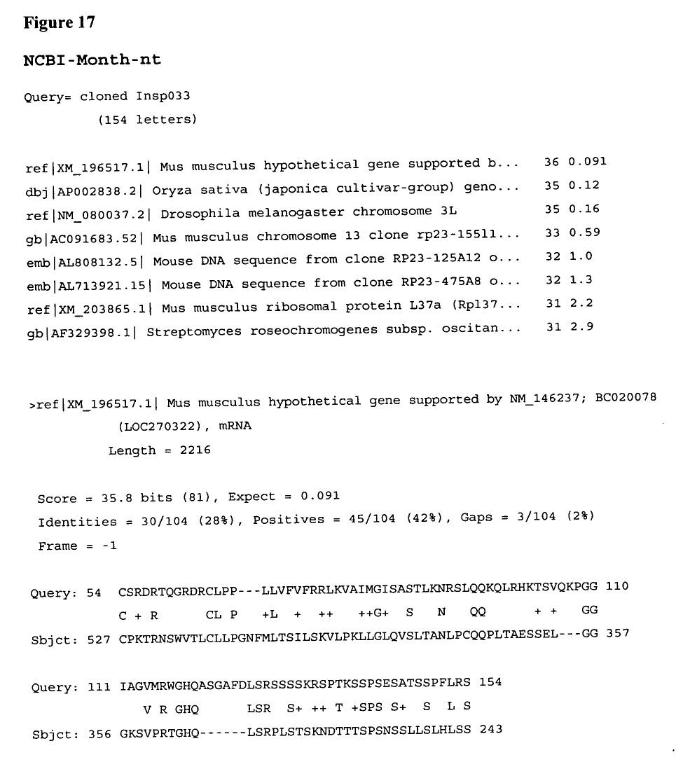

The second stage of the Gateway cloning process involves subcloning of the Gateway modified PCR product into the Gateway entry vector pDONR201 (Invitrogen, FIG. 10) as follows: 5 μl of purified PCR product is incubated with 1.5 μl pDONR201 vector (0.1 μg/μl), 2 μl BP buffer and 1.5 μl of BP clonase enzyme mix (Invitrogen) at RT for 1 h. The reaction was stopped by addition of proteinase K (2 μg) and incubated at 37° C. for a further 10 min. An aliquot of this reaction (2 μl) was transformed into E. coli DH10B cells by electroporation using a Biorad Gene Pulser. Transformants were plated on LB-kanamycin plates. Plasmid mini-prep DNA was prepared from 1-4 of the resultant colonies using Promega Wizard Mini-Prep kits, and 1.5 μl of the plasmid eluate was then used in a recombination reaction containing 1.5 μl pEAKl2d vector (FIG. 11) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 μl) was used to transform E. coli DH10B cells by electroporation.

Clones containing the correct insert were identified by performing colony PCR as described above except that pEAK12d primers (pEAK12d F and pEAK12d R) were used for the PCR. Plasmid mini prep DNA was isolated from clones containing the correct insert using a Qiaprep Turbo 9600 robotic system (Qiagen) or manually, using a Wizard SV DNA minipreps kit (Promega) and sequence verified using the pEAK12d F and pEAK12d R primers.

CsCl gradient purified maxi-prep DNA of plasmid pEAK12d-IPAAA24020-6HIS (plasmid ID number 12156, FIG. 8) was prepared from a 500 ml culture of sequence verified clones (Sambrook J. et al., in Molecular Cloning, a Laboratory Manual, 2^{nd} edition, 1989, Cold Spring Harbor Laboratory Press), resuspended at a concentration of 1 μg/μl in sterile water and stored at −20° C.

2.3 Construction of Expression Vector pEAK12d

The vector pEAK12d is a Gateway Cloning System compatible version of the mammalian cell expression vector pEAK12 (purchased from Edge Biosystems) in which the cDNA of interest is expressed under the control of the human EF1α promoter. pEAK12d was generated as described below:

pEAK12 was digested with restriction enzymes HindIII and NotI, made blunt ended with Klenow (New England Biolabs) and dephosphorylated using calf-intestinal alkaline phosphatase (Roche). After dephosphorylation, the vector was ligated to the blunt ended Gateway reading frame cassette C (Gateway vector conversion system, Invitrogen cat no. 11828-019) which contains AttR recombination sites flanking the ccdB gene and chloramphenicol resistance, and transformed into E.coli DB3.1 cells (which allow propagation of vectors containing the ccdB gene). Mini prep DNA was isolated from several of the resultant colonies using a Wizard SV mini prep kit (Promega) and digested with AseI/EcoRI to identify clones yielding a 670 bp fragment, indicating that the cassette had been inserted in the correct orientation. The resultant plasmid was called pEAK12d (FIG. 11).

2.4 Identification of cDNA Libraries Containing IPAAA24020

PCR products obtained with CP1 and CP2 and migrating at the correct size (538 bp) were identified in the fetal kidney and brain cortex libraries (libraries 12 and 8 respectively).

TABLE I
Human cDNA libraries

| Library | Tissue/cell source | Vector | Host strain | Supplier | Cat. no. |
|---|---|---|---|---|---|
| 1 | human fetal brain | Zap II | XL1-Blue MRF′ | Stratagene | 936206 |
| 2 | human ovary | GT10 | LE392 | Clontech | HL1098a |
| 3 | human pituitary | GT10 | LE392 | Clontech | HL1097a |
| 4 | human placenta | GT11 | LE392 | Clontech | HL1075b |
| 5 | human testis | GT11 | LE392 | Clontech | HL1010b |
| 6 | human sustanta nigra | GT10 | LE392 | in house | |
| 7 | human fetal brain | GT10 | LE392 | in house | |
| 8 | human cortex brain | GT10 | LE392 | in house | |
| 9 | human colon | GT10 | LE392 | Clontech | HL1034a |
| 10 | human fetal brain | GT10 | LE392 | Clontech | HL1065a |
| 11 | human fetal lung | GT10 | LE392 | Clontech | HL1072a |
| 12 | human fetal kidney | GT10 | LE392 | Clontech | HL1071a |
| 13 | human fetal liver | GT10 | LE392 | Clontech | HL1064a |
| 14 | human bone marrow | GT10 | LE392 | Clontech | HL1058a |
| 15 | human peripheral blood monocytes | GT10 | LE392 | Clontech | HL1050a |
| 16 | human placenta | GT10 | LE392 | in house | |
| 17 | human SHSYSY | GT10 | LE392 | in house | |
| 18 | human U373 cell line | GT10 | LE392 | in house | |
| 19 | human CFPoc-1 cell line | Uni Zap | XL1-Blue MRF′ | Stratagene | 936206 |
| 20 | human retina | GT10 | LE392 | Clontech | HL1132a |
| 21 | human urinary bladder | GT10 | LE392 | in house | |
| 22 | human platelets | Uni Zap | XL1-Blue MRF′ | in house | |
| 23 | human neuroblastoma Kan + TS | GT10 | LE392 | in house | |
| 24 | human bronchial smooth muscle | GT10 | LE392 | in house | |
| 25 | human bronchial smooth muscle | GT10 | LE392 | in house | |
| 26 | human Thymus | GT10 | LE392 | Clontech | HL1127a |
| 27 | human spleen 5′ stretch | GT11 | LE392 | Clontech | HL1134b |
| 28 | human peripherical blood monocytes | GT10 | LE392 | Clontech | HL1050a |
| 29 | human testis | GT10 | LE392 | Clontech | HL1065a |
| 30 | human fetal brain | GT10 | LE392 | Clontech | HL1065a |
| 31 | human substancia Nigra | GT10 | LE392 | Clontech | HL1093a |
| 32 | human placenta#11 | GT11 | LE392 | Clontech | HL1075b |
| 33 | human Fetal brain | GT10 | LE392 | Clontech | custom |
| 34 | human placenta #59 | GT10 | LE392 | Clontech | HL5014a |
| 35 | human pituirary | GT10 | LE392 | Clontech | HL1097a |
| 36 | human pancreas #63 | Uni Zap XR | XL1-Blue MRF′ | Stratagene | 937208 |
| 37 | human placenta #19 | GT11 | LE392 | Clontech | HL1008 |
| 38 | human liver 5′ strech | GT11 | LE392 | Clontech | HL1115b |
| 39 | human uterus | Zap-CMV XR | XL1-Blue MRF′ | Stratagene | 980207 |
| 40 | human kidney large-insert cDNA library | TriplEx2 | XL1-Blue | Clontech | HL5507u |

TABLE II
IPAAA24020 Cloning primers

| | | | | | |
|---|---|---|---|---|---|
| Primer | Name | Sequence (5′-3′) | Position | Tm° C. | % GC |

| | | | | | |
|---|---|---|---|---|---|
| CP1 | 3A7 Forward | GCTGCTTCTCCAC | 42 | 64 | 55 |
| | primer | ACCAAGT | | | |
| CP2 | 3A8 Reverse | CACGGAGCCAGTA | 579C | 64 | 55 |
| | Primer | AGCTGAT | | | |

TABLE III

Primers for IPAAA24020 subcloning and
sequencing

| Primer | Name | Sequence (5′-3′) |
|---|---|---|
| GCP | I-C1 attB1-K | G GGG ACA AGT TTG TAC AAA AAA |
| Forward | | GCA GGC TTC GCC ACC |
| GCP | 22A3 attB2- | GGG GAC CAC TTT GTA CAA GAA |
| Reverse | stop-His6-R | AGC TGG GTT TCA ATG GTG ATG |
| | | GTG ATG GTG |
| EX1 | II-A1 | GCA GGC TTC GCC ACC ATG CTT |
| Forward | 24020_B1p | TCC CCG GAC CTC CA |
| EX2 | I-I8 | GTG ATG GTG ATG GTG GGA TCG |
| Reverse | 24020_6HISp | GAG GAA GGG GCT GCT G |
| pEAK12-F | 32D1 | GCC AGC TTG GCA CTT GAT GT |
| pEAK12-R | 32D2 | GAT GGA GGT GGA CGT GTC AG |
| SP6 | | ATT TAG GTG ACA CTA TAG |
| T7 | | TAA TAC GAC TCA CTA TAG GG |

Underlined sequence = Kozak sequence
Bold = Stop codon
Italic sequence = His tag
Shaded sequence = Shine Dalgarno sequence (Ribosome binding site)

3. Expression of IPAAA24020 -6HIS in Mammalian Cells (Plasmid No.12156)
3.1 Culture

Human Embryonic Kidney 293 cells expressing the Epstein-Barr virus Nuclear Antigen (HEK293-EBNA, Invitrogen) were maintained in suspension in Ex-cell VPRO serum-free medium (seed stock, maintenance medium, JRH). Sixteen to 20 hours prior to transfection (Day-1), cells were seeded in 2×T225 flasks (50 ml per flask in DMEM/F12 (1:1) containing 2% FBS (JRH), seeding medium) at a density of 2×10⁵ cells/ml). The next day (transfection day 0) the transfection took place by using the JetPEI™ polymere reagent (PolyPlus-transfection) (2 μl/μg of plasmid DNA). For each flask, 113 μg of plasmid No. 12156 was co-transfected with 2.3 μg of GFP (reporter gene). The transfection mix was then added to the 2×T225 flasks and incubated at 37° C. (5% CO₂) for 6 days. Confirmation of positive transfection was done by fluorescence examination at day 1 and day 6.

On day 6 (harvest day), supernatants (100ml) from the two flasks were pooled and centrifuged (4° C., 400 g) and placed into a pot bearing a unique identifier.

One aliquot (500 ul) was kept for QC of the 6His-tagged protein (internal bioprocessing QC).

3.2 Purification Process

The 100 ml culture medium sample containing the recombinant protein with a C-terminal 6His tag was diluted to a final volume of 200 ml with cold buffer A (50 mM NaH₂PO₄; 600 mM NaCl; 8.7% (w/v) glycerol, pH 7.5). The sample was filtered through a 0.22 um sterile filter (Millipore, 500 ml filter unit) and kept at 4° C. in a 250 ml sterile square media bottle (Nagene).

The purification was performed at 4° C. on the VISION workstation (Applied Biosystems) connected to an automatic sample loader (Labomatic). The purification procedure was composed of two sequential steps, metal affinity chromatography on a Poros 20 MC (Applied Biosystems) column charged with Ni ions (4.6 ×50 mm, 0.83 ml), followed by gel filtration on a Sephadex G-25 medium (Amersham Pharmacia) column (1.0×10 cm).

For the first chromatography step the metal affinity column was regenerated with 30 column volumes of EDTA solution (100 mM EDTA; 1 M NaCl; pH 8.0), recharged with Ni ions through washing with 15 column volumes of a 100 mM NiSO₄ solution, washed with 10 column volumes of buffer A, followed by 7 column volumes of buffer B (50 mM NaH₂PO₄; 600 mM NaCl; 8.7% (w/v) glycerol, 400 mM; imidazole, pH 7.5), and finally equilibrated with 15 column volumes of buffer A containing 15 mM imidazole. The sample was transferred, by the Labomatic sample loader, into a 200 ml sample loop and subsequently charged onto the Ni metal affinity column at a flow rate of 10 ml/min. The column was washed with 12 column volumes of buffer A, followed by 28 column volumes of buffer A containing 20 mM imidazole. During the 20 mM imidazole wash loosely attached contaminating proteins were elution of the column. The recombinant His-tagged protein was finally eluted with 10 column volumes of buffer B at a flow rate of 2 ml/min, and the eluted protein was collected in a 1.6 ml fraction.

For the second chromatography step, the Sephadex G-25 gel-filtration column was regenerated with 2 ml of buffer D (1.137 M NaCl; 2.7 mM KCl; 1.5 mM KH₂PO₄; 8 mM Na₂HPO₄; pH 7.2), and subsequently equilibrated with 4 column volumes of buffer C (137 mM NaCl; 2.7 mM KCl; 1.5 mM KH₂PO₄; 8 mM Na₂HPO₄; 20% (w/v) glycerol; pH 7.4). The peak fraction eluted from the Ni-column was automatically through the integrated sample loader on the VISION loaded onto the Sephadex G-25 column and the protein was eluted with buffer C at a flow rate of 2 ml/min. The desalted sample was recovered in a 2.2 ml fraction. The fraction was filtered through a 0.22 um sterile centrifugation filter (Millipore), frozen and stored at −80 C. An aliquot of the sample was analyzed on SDS-PAGE (4-12% NuPAGE gel; Novex) by Coomassie staining and by Western blot with anti-His antibodies.

The gel for the Western blot analysis was electrotransferred to a nitrocellulose membrane at 290 mA for 1 hour at 4° C. The membrane was blocked with 5% milk powder in buffer E (137 mM NaCl; 2.7 mM KCl; 1.5 mM KH₂PO₄; 8 mM Na₂HPO₄; 0.1% Tween 20, pH 7.4) for 1 h at room temperature, and subsequently incubated with a mixture of 2 rabbit polyclonal anti-His antibodies (G-18 and H-15, 0.2ug/ml each; Santa Cruz) in 2.5% milk powder in buffer E overnight at 4° C. After further 1 hour incubation at room temperature, the membrane was washed with buffer E (3×10 min), and then incubated with a secondary HRP-conjugated anti-rabbit antibody (DAKO, HRP 0399) diluted 1/3000 in buffer E containing 2.5% milk powder for 2 hours at room temperature. After washing with buffer E (3×10 minutes), the membrane was developed with the ECL kit (Amersham Pharmacia) for 1 min. The membrane was subsequently exposed to a Hyperfilm (Amersham Pharmacia), the film developed and the western blot image compared with the Coomassie stained gel.

The protein concentration was determined using the BCA Protein Assay kit (Pierce) with Bovine Serum Albumin as standard in samples that show detectable protein bands by Coomassie staining.

### Example 3 INSP034

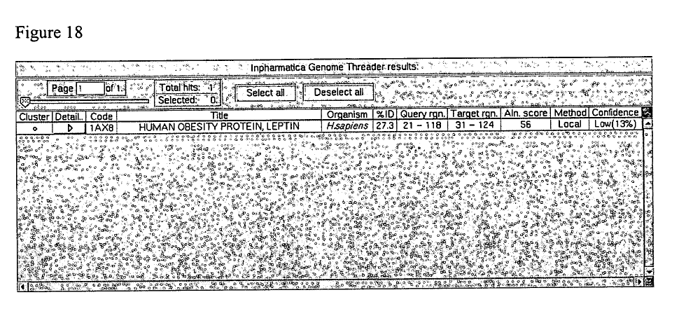

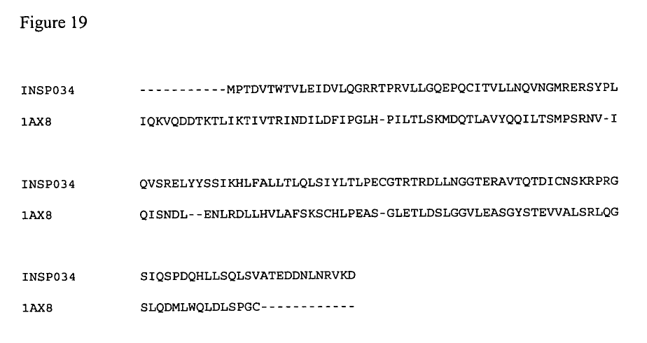

The polypeptide sequence derived from combining SEQ ID NO:10, SEQ ID NO:12 and SEQ ID NO:14 (equivalent to SEQ ID NO:16) which represent the translation of consecutive exons from INSP034 were used as a query in the Inpharmatica Genome Threader tool against protein structures present in the PDB database. The top match is the structure of a four helical bundle cytokine family member. The top match aligns to the query sequence with a Genome Threader confidence of 13% (FIG. 18). FIG. 19 shows the alignment of the INSP034 query sequence to the sequence of human obesity protein (leptin) (PDB-1ax8) a member of the four helical bundle cytokine family (Zhang et al, Nature. 1997 May 8;387(6629):206-9). Members of the four helical bundle cytokine family of secreted proteins are of therapeutic importance.

### Example 4 INSP036

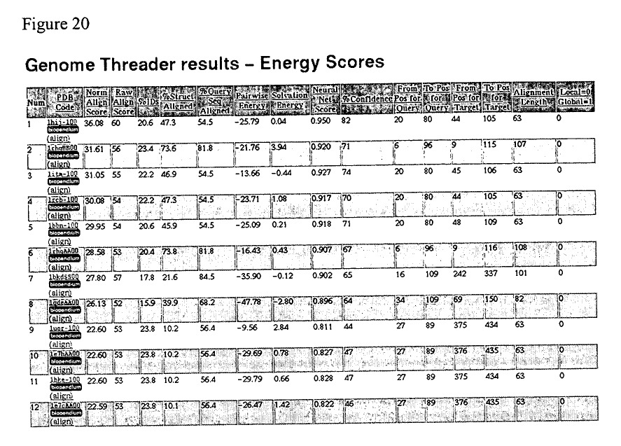

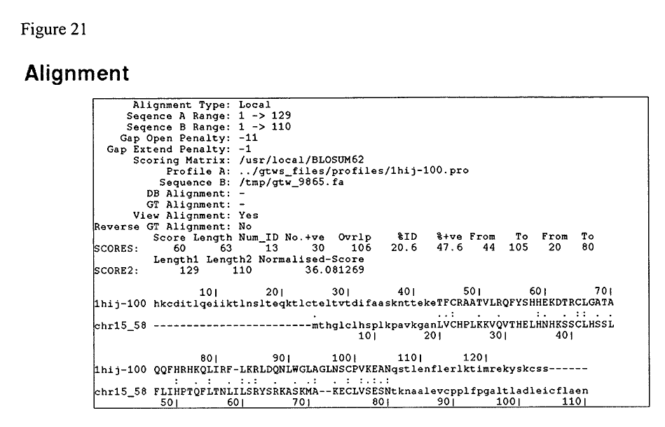

The polypeptide sequence derived from combining SEQ ID NO:24, SEQ ID NO:26, SEQ ID NO:28, SEQ ID NO:30 and SEQ ID NO:32 (equivalent to SEQ ID NO:34) which represent the translation of consecutive exons from INSP036 were used as a query in the Inpharmatica Genome Threader tool against protein structures present in the PDB database. The top match is the structure of a four helical bundle cytokine family member. The top match aligns to the query sequence with a Genome Threader confidence of 82% (FIG. 20). FIG. 21 shows the alignment of the INSP036 query sequence to the sequence of Interleukin-4 (PDB-1hij) a member of the four helical bundle cytokine family (Demchuk et al, Protein Sci. June 1994;3(6):920-35). Note that the INSP036 polypeptide sequence is referred to as “chr15_{—}58” in FIG. 21. Members of the four helical bundle cytokine family of secreted proteins are of therapeutic importance

### Example 5 INSP038

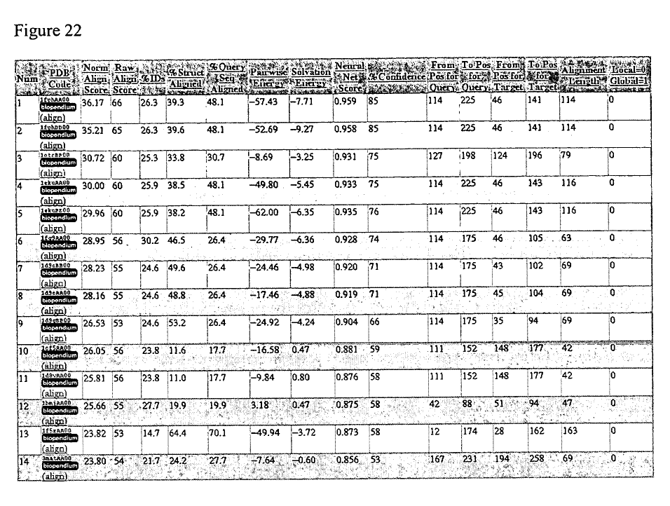

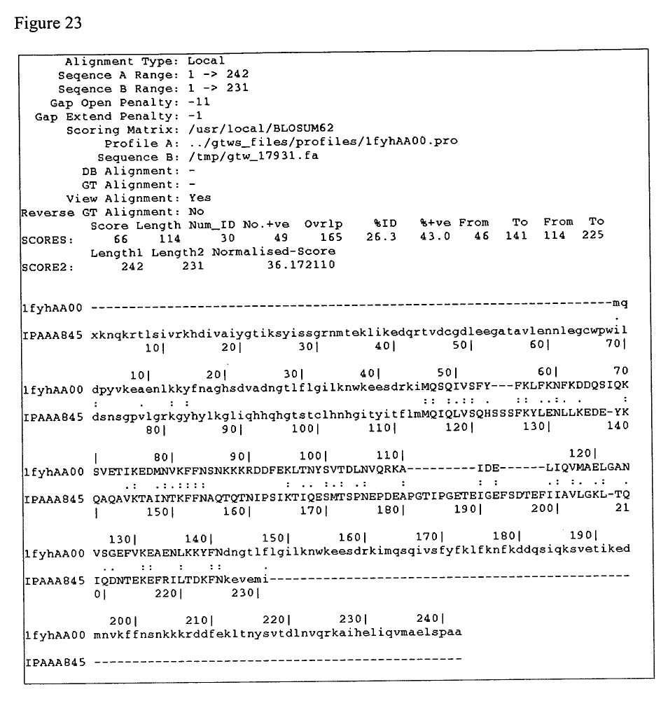

The polypeptide sequence derived from combining SEQ ID NO:38, SEQ ID NO:40, SEQ ID NO:42, SEQ ID NO:44 and SEQ ID NO:46 (equivalent to SEQ ID NO:48) which represent the translation of consecutive exons from INSP038 were used as a query in the Inpharmatica Genome Threader tool against protein structures present in the PDB database. The top matches include structures of four helical bundle cytokine family members. The top match is the structure of a four helical bundle cytokine family member. The top match aligns to the query sequence with a Genome Threader confidence of 85% (FIG. 22). FIG. 23 shows the alignment of the INSP038 query sequence to the sequence of human interferon gamma (PDB-1fyh) a member of the four helical bundle cytokine family (Randal et al, Protein Sci April 1998;7(4):1057-60). Note that the INSP038 polypeptide sequence is referred to as “IPAAA845” in FIG. 23. Members of the four helical bundle cytokine family of secreted proteins are of therapeutic importance

The invention will now be further described by the following numbered paragraphs:

1. A polypeptide, which polypeptide:
(i) comprises or consists of the amino acid sequence as recited in SEQ ID NO:8;
(ii) is a fragment thereof having secreted protein function, four helical bundle cytokine function in particular, or having an antigenic determinant in common with the polypeptides of (i); or
(iii) is a functional equivalent of (i) or (ii).

2. A polypeptide according to paragraph 1, which possesses four helical bundle cytokine function, particularly, possessing granulocyte colony-stimulating factor activity.

3. A polypeptide which is a functional equivalent according to paragraph 1(iii), is homologous to the amino acid sequence as recited in SEQ ID NO:8, and has four helical bundle cytokine activity.

4. A fragment or functional equivalent according to any one of the preceding paragraphs, which has greater than 80% sequence identity with the amino acid sequence recited in SEQ ID NO:8, or with active fragments thereof, preferably greater than 90%, 95%, 98% or 99% sequence identity.

5. A functional equivalent according to any one of the preceding paragraphs, which exhibits significant structural homology with a polypeptide having the amino acid sequence given in SEQ ID NO:8.

6. A fragment as recited in any one of paragraphs 1, 2 or 4 having an antigenic determinant in common with a polypeptide of part (i) of paragraph 1, which consists of 7 or more (for example, 8, 10, 12, 14, 16, 18, 20 or more) amino acid residues from the sequence of SEQ ID NO:8.

7. A purified nucleic acid molecule which encodes a polypeptide according to any one of the preceding paragraphs.

8. A purified nucleic acid molecule according to paragraph 7, which has the nucleic acid sequence as recited in SEQ ID NO:7 or is a redundant equivalent or fragment thereof.

9. A purified nucleic acid molecule which hybridizes under high stringency conditions with a nucleic acid molecule according to paragraph 7 or paragraph 8.

10. A vector comprising a nucleic acid molecule as recited in any one of paragraphs 7-9.

11. A host cell transformed with a vector according to paragraph 10.

12. A ligand which binds specifically to, and which preferably inhibits the secreted protein activity, and in particular four helical bundle cytokine activity of, a polypeptide according to any one of paragraphs 1-6.

13. A ligand according to paragraph 12, which is an antibody.

14. A compound that either increases or decreases the level of expression or activity of a polypeptide according to any one of paragraphs 1-6.

15. A compound according to paragraph 14 that binds to a polypeptide according to any one of paragraphs 1-6 without inducing any of the biological effects of the polypeptide.

16. A compound according to paragraph 14 or paragraph 15, which is a natural or modified substrate, ligand, enzyme, receptor or structural or functional mimetic.

17. A polypeptide according to any one of paragraphs 1-6, a nucleic acid molecule according to any one of paragraphs 7-9, a vector according to paragraph 10, a host cell according to paragraph 11, a host cell according to paragraph 11, a ligand according to paragraph 12 or paragraph 13, or a compound according to any one of paragraphs 14-16, for use in therapy or diagnosis of disease.

18. A method of diagnosing a disease in a patient, comprising assessing the level of expression of a natural gene encoding a polypeptide according to any one of paragraphs 1-6, or assessing the activity of a polypeptide according to any one of paragraphs 1-6, in tissue from said patient and comparing said level of expression or activity to a control level, wherein a level that is different to said control level is indicative of disease.

19. A method according to paragraph 18 that is carried out in vitro.

20. A method according to paragraph 18 or paragraph 19, which comprises the steps of:
(a) contacting a host cell according to paragraph 11 with a biological sample under conditions suitable for the formation of a ligand-polypeptide complex; and (b) detecting said complex.

21. A method according to paragraph 18 or paragraph 19, comprising the steps of:
a) contacting a sample of tissue from the patient with a nucleic acid probe under stringent conditions that allow the formation of a hybrid complex between a nucleic acid molecule according to any one of paragraphs 7-9 and the probe;
b) contacting a control sample with said probe under the same conditions used in step a); and
c) detecting the presence of hybrid complexes in said samples; wherein detection of levels of the hybrid complex in the patient sample that differ from levels of the hybrid complex in the control sample is indicative of disease.

22. A method according to paragraph 18 or paragraph 19, comprising:
a) contacting a sample of nucleic acid from tissue of the patient with a nucleic acid primer under stringent conditions that allow the formation of a hybrid complex between a nucleic acid molecule according to any one of paragraphs 7-9 and the primer;
b) contacting a control sample with said primer under the same conditions used in step a); and
c) amplifying the sampled nucleic acid; and
d) detecting the level of amplified nucleic acid from both patient and control samples; wherein detection of levels of the amplified nucleic acid in the patient sample that differ significantly from levels of the amplified nucleic acid in the control sample is indicative of disease.

23. A method according to paragraph 18 or paragraph 19 comprising:
a) obtaining a tissue sample from a patient being tested for disease;
b) isolating a nucleic acid molecule according to any one of paragraphs 7-9 from said tissue sample; and
c) diagnosing the patient for disease by detecting the presence of a mutation which is associated with disease in the nucleic acid molecule as an indication of the disease.

24. The method of paragraph 23, further comprising amplifying the nucleic acid molecule to form an amplified product and detecting the presence or absence of a mutation in the amplified product.

25. The method of either paragraph 23 or 24, wherein the presence or absence of the mutation in the patient is detected by contacting said nucleic acid molecule with a nucleic acid probe that hybridises to said nucleic acid molecule under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation.

26. A method according to any one of paragraphs 18-25, wherein said disease is selected from cell proliferative disorders, autoimmune/inflammatory disorders, cardiovascular disorders, neurological disorders, developmental disorders, metabolic disorders, infections and other pathological conditions, preferably, immune disorders, such as autoimmune disease, rheumatoid arthritis, osteoarthritis, psoriasis, systemic lupus erythematosus, and multiple sclerosis, inflammatory disorders, such as allergy, rhinitis, conjunctivitis, glomerulonephritis, uveitis, Crohn's disease, ulcerative colitis, inflammatory bowel disease, pancreatitis, digestive system inflammation, sepsis, endotoxic shock, septic shock, cachexia, myalgia, ankylosing spondylitis, myasthenia gravis, post-viral fatigue syndrome, pulmonary disease, respiratory distress syndrome, asthma, chronic-obstructive pulmonary disease, airway inflammation, wound healing, endometriosis, dermatological disease, Behcet's disease, neoplastic disorders, such as melanoma, sarcoma, renal tumour, colon tumour, haematological disease, myeloproliferative disorder, Hodgkin's disease, osteoporosis, obesity, diabetes, gout, cardiovascular disorders, reperfusion injury, atherosclerosis, ischaemic heart disease, cardiac failure, stroke, liver disease, AIDS, AIDS related complex, neurological disorders, male infertility, ageing and infections, including plasmodium infection, bacterial infection and viral infection, particularly human herpesvirus 5 (cytomegalovirus) infection.even more particularly hematological disease, leukocyte disorders including leukopenia, drug-induced leukopenia and leukemia, bone marrow transplantation, bone marrow disease, wound healing, immune disorders such as graft versus host disease and Crohn's disease, neoplastic disorders, melanoma, solid tumour, hyperlipidemia, hyperphosphatemia, anemia, ischemia, stroke, vascular disease, thrombosis, thromboembolism, infarction and infection, most particularly fungal and bacterial infection.

27. Use of a polypeptide according to any one of paragraphs 1-6 as a secreted protein.

28. A pharmaceutical composition comprising a polypeptide according to any one of paragraphs 1-6, a nucleic acid molecule according to any one of paragraphs 7-9, a vector according to paragraph 10, a ligand according to paragraph 12 or paragraph 13, or a compound according to any one of paragraphs 14-16.

29. A vaccine composition comprising a polypeptide according to any one of paragraphs 1-6 or a nucleic acid molecule according to any one of paragraphs 7-9.

30. A polypeptide according to any one of paragraphs 1-6, a nucleic acid molecule according to any one of paragraphs 7-9, a vector according to paragraph 10, a host cell according to paragraph 11, a ligand according to paragraph 12 or paragraph 13, a compound according to any one of paragraphs 14-16, or a pharmaceutical composition according to paragraph 28, for use in the manufacture of a medicament for the treatment of cell proliferative disorders, autoimmune/inflammatory disorders, cardiovascular disorders, neurological disorders, developmental disorders, metabolic disorders, infections and other pathological conditions, preferably, immune disorders, such as autoimmune disease, rheumatoid arthritis, osteoarthritis, psoriasis, systemic lupus erythematosus, and multiple sclerosis, inflammatory disorders, such as allergy, rhinitis, conjunctivitis, glomerulonephritis, uveitis, Crohn's disease, ulcerative colitis, inflammatory bowel disease, pancreatitis, digestive system inflammation, sepsis, endotoxic shock, septic shock, cachexia, myalgia, ankylosing spondylitis, myasthenia gravis, post-viral fatigue syndrome, pulmonary disease, respiratory distress syndrome, asthma, chronic-obstructive pulmonary disease, airway inflammation, wound healing, endometriosis, dermatological disease, Behcet's disease, neoplastic disorders, such as melanoma, sarcoma, renal tumour, colon tumour, haematological disease, myeloproliferative disorder, Hodgkin's disease, osteoporosis, obesity, diabetes, gout, cardiovascular disorders, reperfusion injury, atherosclerosis, ischaemic heart disease, cardiac failure, stroke, liver disease, AIDS, AIDS related complex, neurological disorders, male infertility, ageing and infections, including plasmodium infection, bacterial infection and viral infection, particularly human herpesvirus 5 (cytomegalovirus) infection, even more particularly hematological disease, leukocyte disorders including leukopenia, drug-induced leukopenia and leukemia, bone marrow transplantation, bone marrow disease, wound healing, immune disorders such as graft versus host disease and Crohn's disease, neoplastic disorders, melanoma, solid tumour, hyperlipidemia, hyperphosphatemia, anemia, ischemia, stroke, vascular disease, thrombosis, thromboembolism, infarction and infection, most particularly fungal and bacterial infection.

31. A method of treating a disease in a patient, comprising administering to the patient a polypeptide according to any one of paragraphs 1-6, a nucleic acid molecule according to any one of paragraphs 7-9, a vector according to paragraph 10, a host cell according to paragraph 11, a ligand according to paragraph 12 or paragraph 13, or a compound according to any one of paragraphs 14-16, or a pharmaceutical composition according to paragraph 28.

32. A method according to paragraph 31, wherein, for diseases in which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

33. A method according to paragraph 31, wherein, for diseases in which the expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

34. A method of monitoring the therapeutic treatment of disease in a patient, comprising monitoring over a period of time the level of expression or activity of a polypeptide according to any one of paragraphs 1-6, or the level of expression of a nucleic acid molecule according to any one of paragraphs 7-9 in tissue from said patient, wherein altering said level of expression or activity over the period of time towards a control level is indicative of regression of said disease.

35. A method for the identification of a compound that is effective in the treatment and/or diagnosis of disease, comprising contacting a polypeptide according to any one of paragraphs 1-6, or a nucleic acid molecule according to any one of paragraphs 7-9 with one or more compounds suspected of possessing binding affinity for said polypeptide or nucleic acid molecule, and selecting a compound that binds specifically to said nucleic acid molecule or polypeptide.

36. A kit useful for diagnosing disease comprising a first container containing a nucleic acid probe that hybridises under stringent conditions with a nucleic acid molecule according to any one of paragraphs 7-9; a second container containing primers useful for amplifying said nucleic acid molecule; and instructions for using the probe and primers for facilitating the diagnosis of disease.

37. The kit of paragraph 36, further comprising a third container holding an agent for digesting unhybridised RNA.

38. A kit comprising an array of nucleic acid molecules, at least one of which is a nucleic acid molecule according to any one of paragraphs 7-9.

39. A kit comprising one or more antibodies that bind to a polypeptide as recited in any one of paragraphs 1-6; and a reagent useful for the detection of a binding reaction between said antibody and said polypeptide.

40. A transgenic or knockout non-human animal that has been transformed to express higher, lower or absent levels of a polypeptide according to any one of paragraphs 1-6.

41.A method for screening for a compound effective to treat disease, by contacting a non-human transgenic animal according to paragraph 40 with a candidate compound and determining the effect of the compound on the disease of the animal.

1. A polypeptide, which polypeptide:
(i) comprises or consists of the amino acid sequence as recited in SEQ ID NO:8;
(ii) is a fragment thereof having one or more of secreted protein function, four helical bundle cytokine function , or having an antigenic determinant in common with the polypeptides of (i); or
(iii) is a functional equivalent of (i) or (ii).

2. A polypeptide according to claim 1, which possesses one ore more of four helical bundle cytokine function, or granulocyte colony-stimulating factor activity.

3. A polypeptide which is a functional equivalent according to claim 1(iii), is homologous to the amino acid sequence as recited in SEQ ID NO:8, and has four helical bundle cytokine activity.

4. A fragment or functional equivalent according to claim 1, which has greater than 80% sequence identity with the amino acid sequence recited in SEQ ID NO:8, or with active fragments thereof.

5. A fragment or functional equivalent according to claim 1, which has greater than 90% sequence identity with the amino acid sequence recited in SEQ ID NO:8, or with active fragments thereof.

6. A fragment or functional equivalent according to claim 1, which has greater than 95% sequence identity with the amino acid sequence recited in SEQ ID NO:8, or with active fragments thereof.

7. A fragment or functional equivalent according to claim 1, which has greater than 98% sequence identity with the amino acid sequence recited in SEQ ID NO:8, or with active fragments thereof.

8. A fragment or functional equivalent according to claim 1, which has greater than 99% sequence identity with the amino acid sequence recited in SEQ ID NO:8, or with active fragments thereof.

9. A functional equivalent according to claim 1, which exhibits significant structural homology with a polypeptide having the amino acid sequence given in SEQ ID NO:8.

10. A fragment as recited in claim 1, having an antigenic determinant in common with a polypeptide of part (i) of claim 1, which consists of 7 or more amino acid residues from the sequence of SEQ ID NO:8.

11. The fragment of claim 10, which consists of 8 or more amino acid residues from the sequence of SEQ ID NO:8.

12. The fragment of claim 10, which consists of 10 or more amino acid residues from the sequence of SEQ ID NO:8.

13. The fragment of claim 10, which consists of 12 or more amino acid residues from the sequence of SEQ ID NO:8.

14. The fragment of claim 10, which consists of 14 or more amino acid residues from the sequence of SEQ ID NO:8.

15. The fragment of claim 10, which consists of 16 or more amino acid residues from the sequence of SEQ ID NO:8.

16. The fragment of claim 10, which consists of 18 or more amino acid residues from the sequence of SEQ ID NO:8.

17. The fragment of claim 10, which consists of 20 or more amino acid residues from the sequence of SEQ ID NO:8.

18. A purified nucleic acid molecule which encodes a polypeptide according to claim 1.

19. A purified nucleic acid molecule according to claim 18, which has the nucleic acid sequence as recited in SEQ ID NO:7 or is a redundant equivalent or fragment thereof.

20. A purified nucleic acid molecule which hybridizes under high stringency conditions with a nucleic acid molecule according to claim 18.

21. A vector comprising a nucleic acid molecule as recited in claim 18.

22. A host cell transformed with a vector according to claim 21.

23. A ligand which binds specifically to a polypeptide of claim 1, and which inhibits the secreted protein activity of said polypeptide.

24. A ligand which binds specifically to a polypeptide of claim 1, and which inhibits the four helical bundle cytokine activity of said polypeptide.

25. A ligand according to claim 23, which is an antibody.

26. A compound that either increases or decreases the level of expression or activity of a polypeptide according to claim 1.

27. A compound that either increases or decreases the level of expression or activity of a polypeptide according to claim 1 that binds to a polypeptide according to claim 1 without inducing any of the biological effects of the polypeptide.

28. A compound according to claim 26, which is a natural or modified substrate, ligand, enzyme, receptor or structural or functional mimetic.

29. A polypeptide according to claim 1, a nucleic acid molecule which encodes a polypeptide according to claim 1, a vector comprising said nucleic acid molecule, a host cell transformed with said vector, a ligand which binds specifically to a polypeptide of claim 1, and which inhibits the secreted protein activity of said polypeptide, or a compound that either increases or decreases the level of expression or activity of a polypeptide according to claim 1, for use in therapy or diagnosis of disease.

30. A method of diagnosing a disease in a patient, comprising assessing the level of expression of a natural gene encoding a polypeptide according to claim 1, or assessing the activity of a polypeptide according to claim 1, in tissue from said patient and comparing said level of expression or activity to a control level, wherein a level that is different to said control level is indicative of disease.

31. A method according to claim 30 that is carried out in vitro.

32. A method of diagnosing a disease in a patient, comprising assessing the level of expression of a natural gene encoding a polypeptide according to claim 1, or assessing the activity of a polypeptide according to claim 1, in tissue from said patient and comparing said level of expression or activity to a control level, wherein a level that is different to said control level is indicative of disease, which comprises the steps of: (a) contacting a host cell according to claim 11 with a biological sample under conditions suitable for the formation of a ligand-polypeptide complex; and (b) detecting said complex.

33. A method of diagnosing a disease in a patient, comprising assessing the level of expression of a natural gene encoding a polypeptide according to claim 1, or assessing the activity of a polypeptide according to claim 1, in tissue from said patient and comparing said level of expression or activity to a control level, wherein a level that is different to said control level is indicative of disease, comprising the steps of:
a) contacting a sample of tissue from the patient with a nucleic acid probe under stringent conditions that allow the formation of a hybrid complex between a nucleic acid molecule which encodes a polypeptide according to claim 1 and the probe;
b) contacting a control sample with said probe under the same conditions used in step a); and
c) detecting the presence of hybrid complexes in said samples; wherein detection of levels of the hybrid complex in the patient sample that differ from levels of the hybrid complex in the control sample is indicative of disease.

34. A method of diagnosing a disease in a patient, comprising assessing the level of expression of a natural gene encoding a polypeptide according to claim 1, or assessing the activity of a polypeptide according to claim 1, in tissue from said patient and comparing said level of expression or activity to a control level, wherein a level that is different to said control level is indicative of disease, comprising:
a) contacting a sample of nucleic acid from tissue of the patient with a nucleic acid primer under stringent conditions that allow the formation of a hybrid complex between a nucleic acid molecule which encodes a polypeptide according to claim 1 and the primer;
b) contacting a control sample with said primer under the same conditions used in step a); and
c) amplifying the sampled nucleic acid; and
d) detecting the level of amplified nucleic acid from both patient and control samples; wherein detection of levels of the amplified nucleic acid in the patient sample that differ significantly from levels of the amplified nucleic acid in the control sample is indicative of disease.

35. A method of diagnosing a disease in a patient, comprising assessing the level of expression of a natural gene encoding a polypeptide according to claim 1, or assessing the activity of a polypeptide according to claim 1, in tissue from said patient and comparing said level of expression or activity to a control level, wherein a level that is different to said control level is indicative of disease comprising:
a) obtaining a tissue sample from a patient being tested for disease;
b) isolating a nucleic acid molecule which encodes a polypeptide according to claim 1 from said tissue sample; and
c) diagnosing the patient for disease by detecting the presence of a mutation which is associated with disease in the nucleic acid molecule as an indication of the disease.

36. The method of claim 35, further comprising amplifying the nucleic acid molecule to form an amplified product and detecting the presence or absence of a mutation in the amplified product.

37. The method of either claim 35, wherein the presence or absence of the mutation in the patient is detected by contacting said nucleic acid molecule with a nucleic acid probe that hybridises to said nucleic acid molecule under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation.

38. A method according to claim 30, wherein said disease is selected from cell proliferative disorders, autoimmune/inflammatory disorders, cardiovascular disorders, neurological disorders, developmental disorders, metabolic disorders, infections and other pathological conditions, immune disorders, such as autoimmune disease, rheumatoid arthritis, osteoarthritis, psoriasis, systemic lupus erythematosus, and multiple sclerosis, inflammatory disorders, such as allergy, rhinitis, conjunctivitis, glomerulonephritis, uveitis, Crohn's disease, ulcerative colitis, inflammatory bowel disease, pancreatitis, digestive system inflammation, sepsis, endotoxic shock, septic shock, cachexia, myalgia, ankylosing spondylitis, myasthenia gravis, post-viral fatigue syndrome, pulmonary disease, respiratory distress syndrome, asthma, chronic-obstructive pulmonary disease, airway inflammation, wound healing, endometriosis, dermatological disease, Behcet's disease, neoplastic disorders, such as melanoma, sarcoma, renal tumour, colon tumour, haematological disease, myeloproliferative disorder, Hodgkin's disease, osteoporosis, obesity, diabetes, gout, cardiovascular disorders, reperfusion injury, atherosclerosis, ischaemic heart disease, cardiac failure, stroke, liver disease, AIDS, AIDS related complex, neurological disorders, male infertility, ageing and infections, including plasmodium infection, bacterial infection and viral infection, particularly human herpesvirus 5 (cytomegalovirus) infection.even more particularly hematological disease, leukocyte disorders including leukopenia, drug-induced leukopenia and leukemia, bone marrow transplantation, bone marrow disease, wound healing, immune disorders such as graft versus host disease and Crohn's disease, neoplastic disorders, melanoma, solid tumour, hyperlipidemia, hyperphosphatemia, anemia, ischemia, stroke, vascular disease, thrombosis, thromboembolism, infarction and infection, most particularly fungal and bacterial infection.

39. A method according to claim 32, wherein said disease is selected from cell proliferative disorders, autoimmune/inflammatory disorders, cardiovascular disorders, neurological disorders, developmental disorders, metabolic disorders, infections and other pathological conditions, immune disorders, such as autoimmune disease, rheumatoid arthritis, osteoarthritis, psoriasis, systemic lupus erythematosus, and multiple sclerosis, inflammatory disorders, such as allergy, rhinitis, conjunctivitis, glomerulonephritis, uveitis, Crohn's disease, ulcerative colitis, inflammatory bowel disease, pancreatitis, digestive system inflammation, sepsis, endotoxic shock, septic shock, cachexia, myalgia, ankylosing spondylitis, myasthenia gravis, post-viral fatigue syndrome, pulmonary disease, respiratory distress syndrome, asthma, chronic-obstructive pulmonary disease, airway inflammation, wound healing, endometriosis, dermatological disease, Behcet's disease, neoplastic disorders, such as melanoma, sarcoma, renal tumour, colon tumour, haematological disease, myeloproliferative disorder, Hodgkin's disease, osteoporosis, obesity, diabetes, gout, cardiovascular disorders, reperfusion injury, atherosclerosis, ischaemic heart disease, cardiac failure, stroke, liver disease, AIDS, AIDS related complex, neurological disorders, male infertility, ageing and infections, including plasmodium infection, bacterial infection and viral infection, particularly human herpesvirus 5 (cytomegalovirus) infection.even more particularly hematological disease, leukocyte disorders including leukopenia, drug-induced leukopenia and leukemia, bone marrow transplantation, bone marrow disease, wound healing, immune disorders such as graft versus host disease and Crohn's disease, neoplastic disorders, melanoma, solid tumour, hyperlipidemia, hyperphosphatemia, anemia, ischemia, stroke, vascular disease, thrombosis, thromboembolism, infarction and infection, most particularly fungal and bacterial infection.

40. A method according to claim 33, wherein said disease is selected from cell proliferative disorders, autoimmune/inflammatory disorders, cardiovascular disorders, neurological disorders, developmental disorders, metabolic disorders, infections and other pathological conditions, immune disorders, such as autoimmune disease, rheumatoid arthritis, osteoarthritis, psoriasis, systemic lupus erythematosus, and multiple sclerosis, inflammatory disorders, such as allergy, rhinitis, conjunctivitis, glomerulonephritis, uveitis, Crohn's disease, ulcerative colitis, inflammatory bowel disease, pancreatitis, digestive system inflammation, sepsis, endotoxic shock, septic shock, cachexia, myalgia, ankylosing spondylitis, myasthenia gravis, post-viral fatigue syndrome, pulmonary disease, respiratory distress syndrome, asthma, chronic-obstructive pulmonary disease, airway inflammation, wound healing, endometriosis, dermatological disease, Behcet's disease, neoplastic disorders, such as melanoma, sarcoma, renal tumour, colon tumour, haematological disease, myeloproliferative disorder, Hodgkin's disease, osteoporosis, obesity, diabetes, gout, cardiovascular disorders, reperfusion injury, atherosclerosis, ischaemic heart disease, cardiac failure, stroke, liver disease, AIDS, AIDS related complex, neurological disorders, male infertility, ageing and infections, including plasmodium infection, bacterial infection and viral infection, particularly human herpesvirus 5 (cytomegalovirus) infection.even more particularly hematological disease, leukocyte disorders including leukopenia, drug-induced leukopenia and leukemia, bone marrow transplantation, bone marrow disease, wound healing, immune disorders such as graft versus host disease and Crohn's disease, neoplastic disorders, melanoma, solid tumour, hyperlipidemia, hyperphosphatemia, anemia, ischemia, stroke, vascular disease, thrombosis, thromboembolism, infarction and infection, most particularly fungal and bacterial infection.

41. A method according to claim 34, wherein said disease is selected from cell proliferative disorders, autoimmune/inflammatory disorders, cardiovascular disorders, neurological disorders, developmental disorders, metabolic disorders, infections and other pathological conditions, immune disorders, such as autoimmune disease, rheumatoid arthritis, osteoarthritis, psoriasis, systemic lupus erythematosus, and multiple sclerosis, inflammatory disorders, such as allergy, rhinitis, conjunctivitis, glomerulonephritis, uveitis, Crohn's disease, ulcerative colitis, inflammatory bowel disease, pancreatitis, digestive system inflammation, sepsis, endotoxic shock, septic shock, cachexia, myalgia, ankylosing spondylitis, myasthenia gravis, post-viral fatigue syndrome, pulmonary disease, respiratory distress syndrome, asthma, chronic-obstructive pulmonary disease, airway inflammation, wound healing, endometriosis, dermatological disease, Behcet's disease, neoplastic disorders, such as melanoma, sarcoma, renal tumour, colon tumour, haematological disease, myeloproliferative disorder, Hodgkin's disease, osteoporosis, obesity, diabetes, gout, cardiovascular disorders, reperfusion injury, atherosclerosis, ischaemic heart disease, cardiac failure, stroke, liver disease, AIDS, AIDS related complex, neurological disorders, male infertility, ageing and infections, including plasmodium infection, bacterial infection and viral infection, particularly human herpesvirus 5 (cytomegalovirus) infection.even more particularly hematological disease, leukocyte disorders including leukopenia, drug-induced leukopenia and leukemia, bone marrow transplantation, bone marrow disease, wound healing, immune disorders such as graft versus host disease and Crohn's disease, neoplastic disorders, melanoma, solid tumour, hyperlipidemia, hyperphosphatemia, anemia, ischemia, stroke, vascular disease, thrombosis, thromboembolism, infarction and infection, most particularly fungal and bacterial infection.

42. A method according to claim 35, wherein said disease is selected from cell proliferative disorders, autoimmune/inflammatory disorders, cardiovascular disorders, neurological disorders, developmental disorders, metabolic disorders, infections and other pathological conditions, immune disorders, such as autoimmune disease, rheumatoid arthritis, osteoarthritis, psoriasis, systemic lupus erythematosus, and multiple sclerosis, inflammatory disorders, such as allergy, rhinitis, conjunctivitis, glomerulonephritis, uveitis, Crohn's disease, ulcerative colitis, inflammatory bowel disease, pancreatitis, digestive system inflammation, sepsis, endotoxic shock, septic shock, cachexia, myalgia, ankylosing spondylitis, myasthenia gravis, post-viral fatigue syndrome, pulmonary disease, respiratory distress syndrome, asthma, chronic-obstructive pulmonary disease, airway inflammation, wound healing, endometriosis, dermatological disease, Behcet's disease, neoplastic disorders, such as melanoma, sarcoma, renal tumour, colon tumour, haematological disease, myeloproliferative disorder, Hodgkin's disease, osteoporosis, obesity, diabetes, gout, cardiovascular disorders, reperfusion injury, atherosclerosis, ischaemic heart disease, cardiac failure, stroke, liver disease, AIDS, AIDS related complex, neurological disorders, male infertility, ageing and infections, including plasmodium infection, bacterial infection and viral infection, particularly human herpesvirus 5 (cytomegalovirus) infection.even more particularly hematological disease, leukocyte disorders including leukopenia, drug-induced leukopenia and leukemia, bone marrow transplantation, bone marrow disease, wound healing, immune disorders such as graft versus host disease and Crohn's disease, neoplastic disorders, melanoma, solid tumour, hyperlipidemia, hyperphosphatemia, anemia, ischemia, stroke, vascular disease, thrombosis, thromboembolism, infarction and infection, most particularly fungal and bacterial infection.

43. A pharmaceutical composition comprising a polypeptide according to claim 1, a nucleic acid molecule which encodes a polypeptide according to claim 1, a vector comprising said nucleic acid molecule, a host cell transformed with said vector, a ligand which binds specifically to a polypeptide of claim 1, and which inhibits the secreted protein activity of said polypeptide, or a compound that either increases or decreases the level of expression or activity of a polypeptide according to claim 1.

44. A vaccine composition comprising a polypeptide according to claim 1 or a nucleic acid molecule which encodes a polypeptide according to claim 1.

45. A polypeptide according to claim 1, a nucleic acid molecule which encodes a polypeptide according to claim 1, a vector comprising said nucleic acid molecule, a host cell transformed with said vector, a ligand which binds specifically to a polypeptide of claim 1, and which inhibits the secreted protein activity of said polypeptide, or a compound that either increases or decreases the level of expression or activity of a polypeptide according to claim 1, or a pharmaceutical composition comprising any of the above, for use in the manufacture of a medicament for the treatment of cell proliferative disorders, autoimmune/inflammatory disorders, cardiovascular disorders, neurological disorders, developmental disorders, metabolic disorders, infections and other pathological conditions, immune disorders, such as autoimmune disease, rheumatoid arthritis, osteoarthritis, psoriasis, systemic lupus erythematosus, and multiple sclerosis, inflammatory disorders, such as allergy, rhinitis, conjunctivitis, glomerulonephritis, uveitis, Crohn's disease, ulcerative colitis, inflammatory bowel disease, pancreatitis, digestive system inflammation, sepsis, endotoxic shock, septic shock, cachexia, myalgia, ankylosing spondylitis, myasthenia gravis, post-viral fatigue syndrome, pulmonary disease, respiratory distress syndrome, asthma, chronic-obstructive pulmonary disease, airway inflammation, wound healing, endometriosis, dermatological disease, Behcet's disease, neoplastic disorders, such as melanoma, sarcoma, renal tumour, colon tumour, haematological disease, myeloproliferative disorder, Hodgkin's disease, osteoporosis, obesity, diabetes, gout, cardiovascular disorders, reperfusion injury, atherosclerosis, ischaemic heart disease, cardiac failure, stroke, liver disease, AIDS, AIDS related complex, neurological disorders, male infertility, ageing and infections, including plasmodium infection, bacterial infection and viral infection, particularly human herpesvirus 5 (cytomegalovirus) infection, even more particularly hematological disease, leukocyte disorders including leukopenia, drug-induced leukopenia and leukemia, bone marrow transplantation, bone marrow disease, wound healing, immune disorders such as graft versus host disease and Crohn's disease, neoplastic disorders, melanoma, solid tumour, hyperlipidemia, hyperphosphatemia, anemia, ischemia, stroke, vascular disease, thrombosis, thromboembolism, infarction and infection, most particularly fungal and bacterial infection.

46. A method of treating a disease in a patient, comprising administering to the patient a polypeptide according to claim 1, a nucleic acid molecule which encodes a polypeptide according to claim 1, a vector comprising said nucleic acid molecule, a host cell transformed with said vector, a ligand which binds specifically to a polypeptide of claim 1, and which inhibits the secreted protein activity of said polypeptide, or a compound that either increases or decreases the level of expression or activity of a polypeptide according to claim 1, or a pharmaceutical composition comprising any of the above.

47. A method according to claim 46, wherein, for diseases in which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

48. A method according to claim 46, wherein, for diseases in which the expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

49. A method of monitoring the therapeutic treatment of disease in a patient, comprising monitoring over a period of time the level of expression or activity of a polypeptide according to claim 1, or the level of expression of a nucleic acid molecule which encodes a polypeptide according to claim 1 in tissue from said patient, wherein altering said level of expression or activity over the period of time towards a control level is indicative of regression of said disease.

50. A method for the identification of a compound that is effective in the treatment and/or diagnosis of disease, comprising contacting a polypeptide according to claim 1, or a nucleic acid molecule which encodes a polypeptide according to claim 1 with one or more compounds suspected of possessing binding affinity for said polypeptide or nucleic acid molecule, and selecting a compound that binds specifically to said nucleic acid molecule or polypeptide.

51. A kit useful for diagnosing disease comprising a first container containing a nucleic acid probe that hybridises under stringent conditions with a nucleic acid molecule according to claim 7; a second container containing primers useful for amplifying said nucleic acid molecule; and instructions for using the probe and primers for facilitating the diagnosis of disease.

52. The kit of claim 51, further comprising a third container holding an agent for digesting unhybridised RNA.

53. A kit comprising an array of nucleic acid molecules, at least one of which is a nucleic acid molecule according to claim 18.

54. A kit comprising one or more antibodies that bind to a polypeptide as recited in claim 1; and a reagent useful for the detection of a binding reaction between said antibody and said polypeptide.

55. A transgenic or knockout non-human animal that has been transformed to express higher, lower or absent levels of a polypeptide according to claim 1.

56. A method for screening for a compound effective to treat disease, by contacting a non-human transgenic animal according to claim 55 with a candidate compound and determining the effect of the compound on the disease of the animal.


SEQUENCE LISTING (66 sequences)

SEQ ID NO: 1 - DNA, 150 bp, Homo sapiens
atgctcatggcttactgtaaccactccctggctactgcctatattctgtcaaggccccgaggctctaaatcagcaggtggcaaagtccgccaggcctgtgttcttcccttcagggtggtgaggtcctccaggtcctgtgtgggtcctaag

SEQ ID NO: 2 - protein, 50 aa, Homo sapiens
MLMAYCNHSLATAYILSRPRGSKSAGGKVRQACVLPFRVVRSSRSCVGPK

SEQ ID NO: 3 - DNA, 183 bp, Homo sapiens
gttaaagctgtttgtcatctcaaccacggccttgtcaattgttacaacctcctcatccttgacaaattatttgaagtcattaatgagcttcttgagtgtcttcgtccagatgccatttctacattccttgctgacatctcttcaattccaagcaaggtttttgatacatcttggatgttataa

SEQ ID NO: 4 - protein, 60 aa, Homo sapiens
VKAVCHLNHGLVNCYNLLILDKLFEVINELLECLRPDAISTFLADISSIPSKVFDTSWML

SEQ ID NO: 5 - DNA, 333 bp, Homo sapiens
atgctcatggcttactgtaaccactccctggctactgcctatattctgtcaaggccccgaggctctaaatcagcaggtggcaaagtccgccaggcctgtgttcttcccttcagggtggtgaggtcctccaggtcctgtgtgggtcctaaggttaaagctgtttgtcatctcaaccacggccttgtcaattgttacaacctcctcatccttgacaaattatttgaagtcattaatgagcttcttgagtgtcttcgtccagatgccatttctacattccttgctgacatctcttcaattccaagcaaggtttttgatacatcttggatgttataa

SEQ ID NO: 6 - protein, 110 aa, Homo sapiens
MLMAYCNHSLATAYILSRPRGSKSAGGKVRQACVLPFRVVRSSRSCVGPKVKAVCHLNHGLVNCYNLLILDKLFEVINELLECLRPDAISTFLADISSIPSKVFDTSWML

SEQ ID NO: 7 - DNA, 465 bp, Homo sapiens
atgctttccccggacctccacgaagatgccggaaacttttctgctcaactttgcaaggggccatctaagtgccacttagagacggaagctctgacccaaccccacagggatgttttttcctttcattcctttgctctgaacacttggccgtatgctgtgtgcagcagagacaggactcagggaagagacaggtgcctgccaccactgttggtcttcgtcttccggcgtctgaaagtggcgatcatgggtatatcggccagcactttgaaaaacaggtctctccaacaaaagcaactgagacataaaacatctgtccagaaacctgggggcattgctggtgtcatgaggtggggtcaccaggcgtctggagccttcgacctcagccgcagcagcagcagcaaaagaagccccacaaaatcgtcgccgtccgaatctgcgaccagcagccccttcctccgatcctga

SEQ ID NO: 8 - protein, 154 aa, Homo sapiens [sequence omitted]

SEQ ID NO: 9 - DNA, 130 bp, Homo sapiens
atgcccactgatgtaacctggactgtattggaaattgatgtgctccaaggaaggagaacgcctagggtgctccttggacaagagcctcagtgtattactgtattgctaaatcaagtgaatggaatgcgag

SEQ ID NO: 10 - protein, 44 aa, Homo sapiens
MPTDVTWTVLEIDVLQGRRTPRVLLGQEPQCITVLLNQVNGMRE

SEQ ID NO: 11 - DNA, 168 bp, Homo sapiens
aaaggagctacccacttcaggtctccagagagctgtactacagctcaataaagcacctctttgccttgctcaccctccagttgtccatatacctcactcttcctgaatgcgggacaagaactcgggacctgctgaatggtgggactgaaagagctgtaacacaaacag

SEQ ID NO: 12 - protein, 56 aa, Homo sapiens
RSYPLQVSRELYYSSIKHLFALLTLQLSIYLTLPECGTRTRDLLNGGTERAVTQTD

SEQ ID NO: 13 - DNA, 113 bp, Homo sapiens
atatttgcaacagcaaaaggccacgaggcagcattcagagtccagaccagcatctactatcacagcttagcgtggccactgaagatgacaatttaaatcgtgtgaaggactaa

SEQ ID NO: 14 - protein, 36 aa, Homo sapiens
ICNSKRPRGSIQSPDQHLLSQLSVATEDDNLNRVKD

SEQ ID NO: 15 - DNA, 411 bp, Homo sapiens
atgcccactgatgtaacctggactgtattggaaattgatgtgctccaaggaaggagaacgcctagggtgctccttggacaagagcctcagtgtattactgtattgctaaatcaagtgaatggaatgcgagaaaggagctacccacttcaggtctccagagagctgtactacagctcaataaagcacctctttgccttgctcaccctccagttgtccatatacctcactcttcctgaatgcgggacaagaactcgggacctgctgaatggtgggactgaaagagctgtaacacaaacagatatttgcaacagcaaaaggccacgaggcagcattcagagtccagaccagcatctactatcacagcttagcgtggccactgaagatgacaatttaaatcgtgtgaaggactaa

SEQ ID NO: 16 - protein, 136 aa, Homo sapiens [sequence omitted]

SEQ ID NO: 17-22 - skipped (6 entries)

SEQ ID NO: 23 - DNA, 10 bp, Homo sapiens
atgacacacg

SEQ ID NO: 24 - protein, 4 aa, Homo sapiens
MTHG

SEQ ID NO: 25 - DNA, 87 bp, Homo sapiens
ggctttgtttacacagccccttgaagccagctgtcaagggtgcaaacctggtctgccatcctttgaagaaggttcaggttacacatg

SEQ ID NO: 26 - protein, 29 aa, Homo sapiens
LCLHSPLKPAVKGANLVCHPLKKVQVTHE

SEQ ID NO: 27 - DNA, 101 bp, Homo sapiens
aactgcacaatcataaatccagctgccttcattcctccctcttcctcatccaccccactcaattcttgaccaacttgatactctcaagatacagtagaaag

SEQ ID NO: 28 - protein, 33 aa, Homo sapiens
LHNHKSSCLHSSLFLIHPTQFLTNLILSRYSRK

SEQ ID NO: 29 - DNA, 68 bp, Homo sapiens
gcctcaaagatggcgaaagagtgcctggtttcagagagcaatacgaaaaatgcagctttggaagtctg

SEQ ID NO: 30 - protein, 23 aa, Homo sapiens
ASKMAKECLVSESNTKNAALEVC

SEQ ID NO: 31 - DNA, 67 bp, Homo sapiens
tcctcctctttttcctggtgctcttactcttgcagatttggagatttgttttcttgctgaaaattag

SEQ ID NO: 32 - protein, 21 aa, Homo sapiens
PPLFPGALTLADLEICFLAEN

SEQ ID NO: 33 - DNA, 333 bp, Homo sapiens
atgacacacgggctttgtttacacagccccttgaagccagctgtcaagggtgcaaacctggtctgccatcctttgaagaaggttcaggttacacatgaactgcacaatcataaatccagctgccttcattcctccctcttcctcatccaccccactcaattcttgaccaacttgatactctcaagatacagtagaaaggcctcaaagatggcgaaagagtgcctggtttcagagagcaatacgaaaaatgcagctttggaagtctgtcctcctctttttcctggtgctcttactcttgcagatttggagatttgttttcttgctgaaaattag

SEQ ID NO: 34 - protein, 110 aa, Homo sapiens [sequence omitted]

SEQ ID NO: 35-36 - skipped (2 entries)

SEQ ID NO: 37 - DNA, 123 bp, Homo sapiens
aaaaatcagaaaagaacactctccatcgtcagaaagcatgacattgttgccatatatggaacaattaaaagttatatctcatctggaagaaatatgacggaaaaattaataaaggaggaccag

SEQ ID NO: 38 - protein, 41 aa, Homo sapiens
KNQKRTLSIVRKHDIVAIYGTIKSYISSGRNMTEKLIKEDQ

SEQ ID NO: 39 - DNA, 31 bp, Homo sapiens
agaactgtggattgtggggatttagaagaag

SEQ ID NO: 40 - protein, 11 aa, Homo sapiens
RTVDCGDLEEG

SEQ ID NO: 41 - DNA, 40 bp, Homo sapiens
gtgctacagcagttctggaaaacaacttggaaggctgttg

SEQ ID NO: 42 - protein, 13 aa, Homo sapiens
ATAVLENNLEGCW

SEQ ID NO: 43 - DNA, 65 bp, Homo sapiens
gccttggattttggacagcaattctggacctgtcctgggccggaagggatatcactacctgaaag

SEQ ID NO: 44 - protein, 22 aa, Homo sapiens
PWILDSNSGPVLGRKGYHYLKG

SEQ ID NO: 45 - DNA, 434 bp, Homo sapiens
gtcttatccaacatcaccaacatggtacctccacatgtctgcacaatcatggaattacttatattacattcctgatgatgcagatacaacttgtatcacaacactcaagttcttttaaatacctggaaaaccttctcaaggaggatgagtacaaacaagcccaggctgtgaagactgcaataaataccaaattcttcaatgcccagacacagacaaacataccaagtatcaagaccatccaggaaagcatgacctcaccaaatgaaccggatgaagcaccagggaccattcctggagaaacagagataggtgaattttcagacacagaattcataatagctgttttggggaaactcacacaaattcaagataacacagagaaggaattcagaattctaacagacaaatttaataaagaggttgagatgatttaa

SEQ ID NO: 46 - protein, 143 aa, Homo sapiens
LIQHHQHGTSTCLHNHGITYITFLMMQIQLVSQHSSSFKYLENLLKEDEYKQAQAVKTAINTKFFNAQTQTNIPSIKTIQESMTSPNEPDEAPGTIPGETEIGEFSDTEFIIAVLGKLTQIQDNTEKEFRILTDKFNKEVEMI

SEQ ID NO: 47 - DNA, 694 bp, Homo sapiens
gaaaaatcagaaaagaacactctccatcgtcagaaagcatgacattgttgccatatatggaacaattaaaagttatatctcatctggaagaaatatgacggaaaaattaataaaggaggaccagagaactgtggattgtggggatttagaagaaggtgctacagcagttctggaaaacaacttggaaggctgttggccttggattttggacagcaattctggacctgtcctgggccggaagggatatcactacctgaaaggtcttatccaacatcaccaacatggtacctccacatgtctgcacaatcatggaattacttatattacattcctgatgatgcagatacaacttgtatcacaacactcaagttcttttaaatacctggaaaaccttctcaaggaggatgagtacaaacaagcccaggctgtgaagactgcaataaataccaaattcttcaatgcccagacacagacaaacataccaagtatcaagaccatccaggaaagcatgacctcaccaaatgaaccggatgaagcaccagggaccattcctggagaaacagagataggtgaattttcagacacagaattcataatagctgttttggggaaactcacacaaattcaagataacacagagaaggaattcagaattctaacagacaaatttaataaagaggttgagatgatttaa

SEQ ID NO: 48 - protein, 230 aa, Homo sapiens
KNQKRTLSIVRKHDIVAIYGTIKSYISSGRNMTEKLIKEDQRTVDCGDLEEGATAVLENNLEGCWPWILDSNSGPVLGRKGYHYLKGLIQHHQHGTSTCLHNHGITYITFLMMQIQLVSQHSSSFKYLENLLKEDEYKQAQAVKTAINTKFFNAQTQTNIPSIKTIQESMTSPNEPDEAPGTIPGETEIGEFSDTEFIIAVLGKLTQIQDNTEKEFRILTDKFNKEVEMI

SEQ ID NO: 49 - DNA, 20 bp, Artificial Sequence
  3A7 forward primer
gctgcttctccacaccaagt

SEQ ID NO: 50 - DNA, 20 bp, Artificial Sequence
  3A8 reverse primer
cacggagccagtaagctgat

SEQ ID NO: 51 - DNA, 37 bp, Artificial Sequence
  GCP forward primer
ggggacaagtttgtacaaaaaagcaggcttcgccacc

SEQ ID NO: 52 - DNA, 51 bp, Artificial Sequence
  GCP reverse primer
ggggaccactttgtacaagaaagctgggtttcaatggtgatggtgatggtg

SEQ ID NO: 53 - DNA, 35 bp, Artificial Sequence
  EX1 forward primer
gcaggcttcgccaccatgctttccccggacctcca

SEQ ID NO: 54 - DNA, 37 bp, Artificial Sequence
  EX2 reverse primer
gtgatggtgatggtgggatcggaggaaggggctgctg

SEQ ID NO: 55 - DNA, 20 bp, Artificial Sequence
  pEAK12-F primer
gccagcttggcacttgatgt

SEQ ID NO: 56 - DNA, 20 bp, Artificial Sequence
  pEAK12-R primer
gatggaggtggacgtgtcag

SEQ ID NO: 57 - DNA, 18 bp, Artificial Sequence
  SP6 primer
atttaggtgacactatag

SEQ ID NO: 58 - DNA, 20 bp, Artificial Sequence
  T7 primer
taatacgactcactataggg

SEQ ID NO: 59 - protein, 158 aa, Bos taurus
SLPQSFLLKCLEQVRKIQADGAELQERLCAAHKLCHPEELMLLRHSLGIPQAPLSSCSSQSLQLRGCLNQLHGGLFLYQGLLQALAGISPELAPTLDTLQLDVTDFATNIWLQMEDLGAAPAMPTFTSAFQRRAGGVLVASQLHRFLELAYRGLRYLA

SEQ ID NO: 60 - DNA, 4489 bp, Artificial Sequence
  PCRII-TOPO-IPAAA24020
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgcccttcacggagccagtaagctgatcaatgagtcaggatcggaggaaggggctgctggtcgcagattcggacggcgacgattttgtggggcttcttttgctgctgctgctgcggctgaggtcgaaggctccagacgcctggtgaccccacctcatgacaccagcaatgcccccaggtttctggacagatgttttatgtctcagttgcttttgttggagagacctgtttttcaaagtgctggccgatatacccatgatcgccactttcagacgccggaagacgaagaccaacagtggtggcaggcacctgtctcttccctgagtcctgtctctgctgcacacagcatacggccaagtgttcagagcaaaggaatgaaaggaaaaaacatccctgtggggttgggtcagagcttccgtctctaagtggcacttagatggccccttgcaaagttgagcagaaaagtttccggcatcttcgtggaggtccggggaaagcattcgcttcttgactcaccattgcagtaaacttggtgtggagaagcagcaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaattcagggcgcaagggctgctaaaggaaccggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcgccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgatccatggcgatgcctgcttgccgaatatcatggtggaaaatggccgcttttctggattcaacgactgtggccggctgggtgtggcggaccgctatcaggacatagcgttggatacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtgatacactattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactgaatggaggcggataaagttgcaggaccacttctgcgctcggcccttccggctggctggtttattgctgataaatctggagccggtgagcgtgggtctcgcggtatcattgcagcactggggccagatggtaagcgctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 61 - DNA, 7429 bp, Artificial Sequence
  pEAK12D-IPAAA24020-6HIS
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgcatgttcggcgaggcggggcctgcgagcgcggccaccgagaatcggacgggggtagtctcaagctggccggcctgctctggtgcctggcctcgcgccgccgtgtatcgccccgccctgggcggcaaggctgggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaattaatacgactcactatagggagacttctttctcccatttcaggtgtcgtaagctatcaaacaagtttgtacaaaaaagcaggcttcgccaccatgctttccccggacctccacgaagatgccggaaacttttctgctcaactttgcaaggggccatctaagtgccacttagagacggaagctctgacccaaccccacagggatgttttttcctttcattcctttgctctgaacacttggccgtatgctgtgtgcagcagagacaggactcagggaagagacaggtgcctgccaccactgttggtcttcgtcttccggcgtctgaaagtggcgatcatgggtatatcggccagcactttgaaaaacaggtctctccaacaaaagcaactgagacataaaacatctgtccagaaacctgggggcattgctggtgtcatgaggtggggtcaccaggcgtctggagccttcgacctcagccgcagcagcagcagcaaaagaagccccacaaaatcgtcgccgtccgaatctgcgaccagcagccccttcctccgatcccaccatcaccatcaccattgaaacccagctttcttgtacaaagtggttcgatggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatccgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccggcacctcctccagccccagcacctccaccagccccagctcccccagctccagccccaccagcaccagcccctccagccccaccagccccagcccctccggcacctcctccagccccagcacctccaccagccccagctcccccagctccagccccaccagcaccagcccctccagccccaccagccccagcccctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaagctaagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattc

SEQ ID NO: 62 - protein, 130 aa, Homo sapiens
IQKVQDDTKTLIKTIVTRINDILDFIPGLHPILTLSKMDQTLAVYQQILTSMPSRNVIQISNDLENLRDLLHVLAFSKSCHLPEASGLETLDSLGGVLEASGYSTEVVALSRLQGSLQDMLWQLDLSPGC

SEQ ID NO: 63 - protein, 129 aa, Homo sapiens
HKCDITLQEIIKTLNSLTEQKTLCTELTVTDIFAASKNTTEKETFCRAATVLRQFYSHHEKDTRCLGATAQQFHRHKQLIRFLKRLDQNLWGLAGLNSCPVKEANQSTLENFLERLKTIMREKYSKCSS

SEQ ID NO: 64 - protein, 242 aa, Homo sapiens
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELGANVSGEFVKEAENLKKYFNDNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAA

SEQ ID NO: 65 - DNA, 483 bp, Homo sapiens
atggtgagtcaagaagcgaatctttccccggacctccacgaagatgccggaaacttttctgctcaactttgcaaggggccatctaagtgccacttagagacggaagctctgacccaaccccacagggatgttttttcctttcattcctttgctctgaacacttggccgtatgctgtgtgcagcagagacaggactcagggaagagacaggtgcctgccaccactgttggtcttcgtcttccggcgtctgaaagtggcgatcatgggtatatcggccagcactttgaaaaacaggtctctccaacaaaagcaactgagacataaaacatctgtccagaaacctgggggcattgctggtgtcatgaggtggggtcaccaggcgtctggagccttcgacctcagccgcagcagcagcagcaaaagaagccccacaaaatcgtcgccgtccgaatctgcgaccagcagccccttcctccgatcctga

SEQ ID NO: 66 - protein, 160 aa, Homo sapiens
MVSQEANLSPDLHEDAGNFSAQLCKGPSKCHLETEALTQPHRDVFSFHSFALNTWPYAVCSRDRTQGRDRCLPPLLVFVFRRLKVAIMGISASTLKNRSLQQKQLRHKTSVQKPGGIAGVMRWGHQASGAFDLSRSSSSKRSPTKSSPSESATSSPFLRS